(19) United States
(12) Patent Application Publication
(10) Pub. No.: US 2011/0312872 A1
(43) Pub. Date: Dec. 22, 2011

(54) NORRIN IN THE TREATMENT OF DISEASES ASSOCIATED WITH AN INCREASED TGF-BETA ACTIVITY

(75) Inventors: Ernst Tamm, Regensburg (DE); Andreas Ohlmann, Munchen (DE)

(73) Assignee: UNIVERSITÄT REGENSBURG

(21) Appl. No.: 13/141,047

(22) PCT Filed: Dec. 18, 2009
(86) PCT No.: PCT/EP2009/067553

§ 371 Date: Sep. 6, 2011

(30) Foreign Application Priority Data
Dec. 22, 2008 (EP) 08022292.0

(51) Int. Cl. A61K 38/17; C07K 19/00; A61P 43/00; A61P 11/00; A61P 11/06; A61P 1/18; A61P 27/02; A61P 9/00; A61P 13/12; A61P 29/00; A61P 17/00; A61P 17/02; A61P 27/06; A61P 35/00; C07K 14/47

(52) U.S. Cl. 514/1.5; 530/350; 530/387.3; 514/8.9; 514/1.8; 514/1.7

(57) ABSTRACT

The present invention relates to Norrin or a functional fragment thereof in the treatment or prevention of diseases associated with an increased TGF-beta activity. In particular, the use of said Norrin or functional fragment thereof to treat fibrotic diseases/disorders or proliferative disorders, like cancers, is part of this invention.

The present invention relates to Norrin or a functional fragment thereof in the treatment or prevention of diseases associated with an increased TGF-beta activity. In particular, the use of Norrin or a functional fragment thereof to treat fibrotic diseases/disorders or proliferative disorders, like cancers, is part of this invention.

The TGF (Transforming Growth Factor)-β superfamily includes various forms of TGF-β (also known as TGF-beta), inter alia, TGF-β1, TGF-β2 and other TGF-β forms, bone morphogenic protein, nodals, activin, the anti-Mullerian hormone, and other factors. The family has similar signaling pathways, an overlap of biological effects and shares a common structure. In mammals, three TGF-β isoforms with a similar peptide structure exist, namely TGF-β1, TGF-β2 and TGF-β3.

TGF-β1 and TGF-β2 play a central role in the regulation of vital homeostatic processes of an organism, such as the modulation of the immune system, the regulation of cell growth and of cell death or the regulation of the turn-over of the extracellular matrix. TGF-β induces the expression of extracellular matrix proteins in mesenchymal cells and stimulates the production of protease inhibitors which prevent enzymatic breakdown of the matrix.

An increased activity of TGF-β1, TGF-β2 or further TGF-β species is often pathogenic. For example, TGF-β is a crucial factor in the pathogenesis of fibrotic diseases which are associated with a pathologic proliferation and changes in the structure of the extracellular matrix; see Ihn (2002), Curr Opin Rheumatol 14, 681-685. Enhanced expression of TGF-β has been demonstrated in fibrotic tissues, and in particular in systemic sclerosis. Fibrotic diseases associated with increased expression of TGF-β which have been described in the art are, inter alia, scleroderma (Ihn (2002), loc cit.), lung fibrosis (Willis (2007), Am J Physiol Lung Cell Mol Physiol 293(3), L525-34), liver cirrhosis (Gressner (2006), J Cell Mol Med 10, 76-99), glomerulosclerosis of the kidneys (Schnaper (2003) Am J Physiol Renal Physiol 284, F243-F252), and glaucoma (Lutjen-Drecoll (2005), Exp Eye Res 81, 1-4).

High amounts of TGF-β2 are found in the central nervous system (CNS) where TGF-β2 acts as suppressor of the cellular immune response, thus effecting a rapid growth of certain malignant tumors of the CNS.

In the art, inhibition of TGF-β or of TGF-β synthesis has been described in the treatment of diseases known to be associated with increased TGF expression. For example, inhibition of the synthesis of TGF-β2 may inhibit growth of these tumors and is used in cancer therapy; see Rich (2003), Front Biosc 8, e245-260.

In context of the role of increased TGF-β expression in renal disease (glomerular or renal fibrosis), Schnaper (2003; loc. cit.) describes that inhibition of TGF-β binding to its receptor can lessen the degree of experimental renal fibrosis. However, Schnaper is predominantly interested in inhibition of TGF-β signaling. Elevated TGF-β signaling has also been described in the art to inhibit ocular vascular development; see Zhao (2001) Dev Biol 237, 45-53. Flügel-Koch ((2002) Dev Dyn 225, 111-25) disclose that TGF-β1 overexpression in murine eyes may also lead to a disruption of anterior segment development.

Willis (2007; loc. cit.) describes the use of certain compounds in reverting TGF-β1-induced epithelial-mesenchymal transition (EMT). EMT is a process which involves the transition of fully differentiated epithelial cells to a mesenchymal phenotype, thus giving rise to fibroblasts and myofibrolasts. It is speculated in the art that this process contributes to fibrosis following injury in the lung. Willis describes that BMP-7 (Bone morphogenic protein-7) reverts TGF-β1-induced EMT in adult tubular epithelial cells by directly counteracting TGF-β1-induced Smad3-dependent EMT and may, accordingly, be used in the treatment of renal fibrosis. Also the hepatocyte growth factor (HGF1) is described in Willis to block EMT by upregulating the Smad transcriptional co-repressor SnoN which leads to the formation of a transcriptionally inactive SnoN/Smad complex and thereby blocks the effects of TGF-β1. In sum, Willis proposes modulating Smad activity (as part of the TGF-β-signaling pathway) as strategy for counteracting TGF-β-induced EMT.

Gessner (2006; loc. cit.) speculates that TGF-β may be an important player in liver fibrosis and proposes TGF-β as target for potential therapies of fibrosis. Gessner provides a list of agents which may be used in the treatment of fibrosis, whereby some compounds interfere with gene expression and synthesis of extracellular matrix (ECM) components while other compounds affect the deposition of fibrillar ECM, reduce the pro-fibrogenic effects of reactive oxygen species (ROS) or have miscellaneous effects on hepatic stellate cells (HSC), a major fibrogenic liver cell type. Also described are approaches aiming to sequester TGF-β or its synthesis. For example, Gessner (2006; loc. cit.) proposes the application of soluble or dominant negative receptors against TGF-β, thus inhibiting TGF-β mediated signaling. Also the use of antisense technology in blocking the synthesis of TGF-β, the use of mono- or polyclonal neutralizing antibodies, inhibition of proteins necessary to release biological TGF-β from its precursor or latent complexes, the utilization of TGF-β sequestering proteins such as α₂-macroglobulin or decorin is proposed. Also small molecules (e.g. SB-431542, SB-505124, SE-208, A-83-01) are described as inhibitors of TβRI subspecies.

It is recognized in the art that there is a need to identify compounds which can be used in the treatment of diseases with aberrant TGF-β expression. Accordingly, the technical problem underlying the present invention is the provision of means and methods to treat diseases with an aberrant TGF-β expression.

The technical problem is solved by provision of the embodiments characterized in the claims.

Accordingly, the present invention relates to Norrin or a functional fragment thereof for use in treating or preventing a disease associated with an increased TGF-β activity. In an alternative embodiment, the present invention relates to the use of Norrin or a functional fragment thereof for the preparation of a pharmaceutical composition for the treatment or prevention of a disease associated with an increased TGF-β activity.

Norrin has been described in the art as protein involved in retinal development, whereby mutations in the gene encoding Norrin may lead to abnormalities in said development, resulting eventually in retinal degeneration and blindness. The gist of the present invention lies in the surprising identification of Norrin as a potent TGFβ-antagonist/inhibitor. It was unexpectedly found in the present invention that Norrin (or a functional fragment thereof) may, therefore, be used in the treatment or prevention of a disease associated with an increased TGF-β activity.

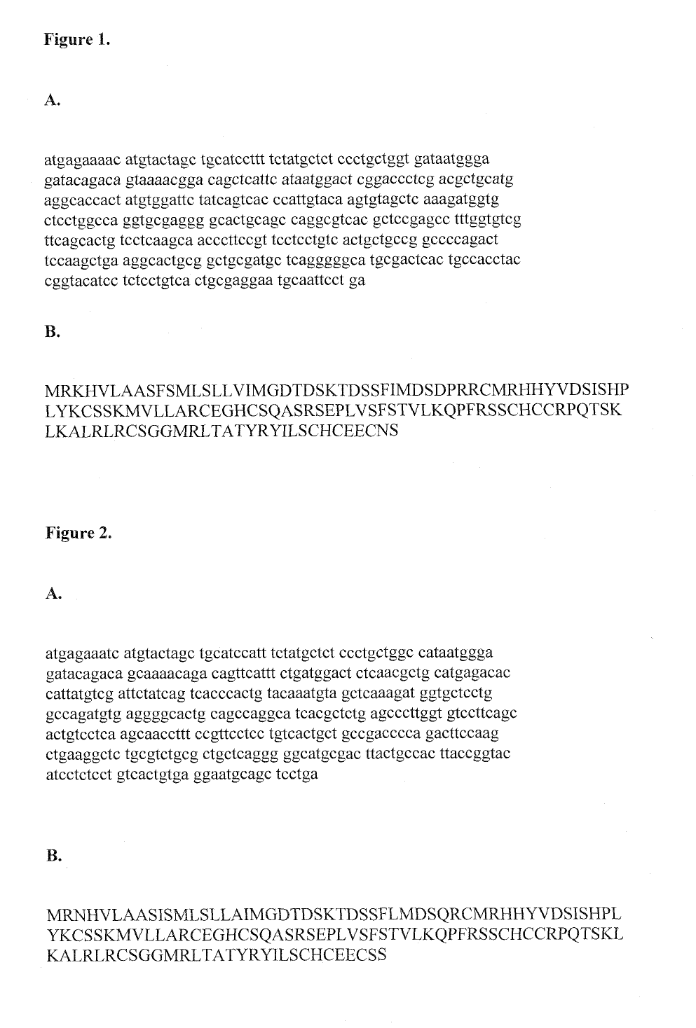

Human Norrin (also known as Norrie disease (pseudoglioma) or NDP protein) is a secreted protein of about 133 amino acids, whereas the murine Norrin ortholog has a length of about 131 amino acids. The nucleic acid sequence and amino acid sequence of human and murine Norrin are shown in SEQ ID NOs: 1 and 2 (FIG. 1) and SEQ ID NOs: 3 and 4 (FIG. 2), respectively. Norrin is thought of being primarily involved in the Wnt signaling pathway and described herein below in more detail.

Human Norrin/NDP protein has been identified in the art as being a crucial factor in the development of Norrie disease. It is known that mutations in the gene encoding NDP can lead to the Norrie disease which is characterized by congenital or infantile blindness, deafness and retarded mental development; see Berger (1998), Acta Anat 162, 95-100. Norrie disease patients show proliferative and degenerative changes in the vitreous body and retina and the formation of retrolental proliferated tissue (pseudoglioma); see Warburg (1961), Acta Ophtalmol 39, 757-772; Warburg (1963), Acta Ophtalmol 41, 134-146.

In about 20% of patients suffering from Norrie disease deletions of between two kilobasepairs to several hundred kilobasepairs in the NDP gene locus are found; see de la Chapelle (1985), Clin Genet 28, 317-320; Donnai (1988), J Med Genet 25, 73-78; Gal (1985), Clin Genet 27, 282-283; Gal (1986), Cytogenet Cell Genet 42, 219-224; Zhu (1989), Am J Med Genet 33, 485-488. Also point mutations in the NDP gene have been described which lead to the synthesis of a truncated or elongated gene product or exchange of amino acids; see Berger (1992), Hum Mol Genet 1, 461-465; Chen (1993), Nat Genet 5, 180-183; Fuchs (1994), Hum Mol Genet 3, 655-656; Fuentes (1993), Hum Mol Genet 2, 1953-1955; Wong (1993), Arch Ophtalmol 111, 1553-1557. The NDP gene consists of three exons and has a length of about 28 kilobasepairs (kbp). The length of the transcript is about 1.9 kbp, whereby exon 2 and 3 comprise the coding sequences for Norrin.

The orthologous murine Norrin/NDP gene is highly conserved and encodes a protein having 94% homology to human Norrin/NDP; see Battinelli (1996), Mamm Genome 7, 93-97. The Norrin gene is predominantly expressed in the brain and the retina; see Berger (1996), Hum Mol Genet 5, 51-59. Norrin mRNA is found in the inner nuclear layer as well as in the retinal ganglion layer of the eye; see Berger (1996, loc. cit.); Hartzer (1999), Brain Res Bull 49, 355-358. Expression of the Norrin gene starts in the late fetal phase and persists in the adult eye, where Norrin can be detected in the central and peripheral retina; see Berger (1996, loc. cit.), Hartzer (1999, loc. cit.), Bernstein (1998), Mol Vis 4, 24.

In sum, Norrin has only been described in the art as protein involved in retinal development. In contrast thereto, Norrin has been identified in the present invention for the first time as a potent TGF-β antagonist/inhibitor; see also the appended example. Furthermore, it has been surprisingly found herein that Norrin can be used in the treatment of (a) disease(s) which is (are) associated with an increased TGF-β activity, such as (a) fibrotic disease(s) or (a) proliferative disease(s) as described herein below in more detail.

Norrin has not been described in the art as antagonist or inhibitor of TGF-β and has not even been mentioned as being capable of interfering with the TGF-β signaling pathway. As mentioned above, TGF-β inhibitors/antagonists described or proposed in the art are thought to interfere with components of the TGF-β signaling pathway, such as TGF-β itself or Smad proteins; see, for example, Gressner (2006), loc. cit.; Willis (2007), loc. cit; Schnaper (2003), loc. cit. In contrast thereto, Norrin is known to bind to the Frizzled-4 receptor (a receptor of the Wnt-signaling pathway) and is thought of being capable of activating the classical Wnt-signaling pathway; see Xu (2004), Cell 116, 883-895. However, a role of Norrin as TGF-β antagonist has not been described in the prior art.

The biochemical function of Norrin has not been elucidated in detail in the art. Norrin is merely known to be a secreted protein which forms oligomers via disulfide bonds. These oligomers are associated with the extracellular matrix; see Perez-Vilar (1997), J Biol Chem 272, 33410-33415. The amino acid sequence of Norrin is partially homologous to the cystein-rich domains of mucins and proteins which are involved in cellular interactions and differentiation processes; see Meindl (1992), Nat Genet 2, 139-143. However, no function has been assigned so far to the homologous C-terminal cysteine-rich domain (CT-domain). The highest identity and similarity values are found between Norrin and the human intestinal mucin (MUC2) with values of 30% and 49%, respectively. A comparison of Norrin and TGF-β showed non-significant values of at most 10% for identity and 25% for similarity; see Meitinger (1993), Nat Genet 5, 376-380.

Molecular modelling proposes a tertiary structure of Norrin similar to Transforming Growth Factor-β and other growth factors (such as nerve growth factor (NGF) and platelet derived growth factor (PDGF-B) with a “cysteine-knot” motif. Yet, the molecular model has not been validated experimentally. However, the prior art does not speculate or describe whether Norrin and other growth factors have a similar biochemical function. The above-mentioned cysteine-knot motif (consisting of six cysteine residues) is essentially the only conserved feature when the primary sequences of the different growth factors are compared. The cysteins of the cysteine-knot motif are thought of being involved in forming disulfide bridges; see Meitinger (1993, loc. cit).

Even though the biochemical function of Norrin has not been disclosed in detail, a role of Norrin in retinal angiogenesis is assumed in the art. For example, mice with a deletion of the gene coding for NDP do not develop capillaries in the retina and in the Stria vascularis of the inner ear and show an increased loss of retinal ganglion cells; see Richter (1998), Invest Ophtalmol Vis Sci 39, 2450-2457, Rehm (2002), J Neurosci 22, 4286-4292. Vice versa, an increased expression of Norrin in murine eyes leads to an increased formation of capillaries and an increase of retinal neurons; see Ohlmann (2005), J Neurosci 25, 1701-1710. The ectopic expression of Norrin in the lens of transgenic animals and subsequent secretion from the lens is sufficient to normalize the retinal phenotype of Norrin-deficient mice; see Ohlmann (2005; loc. cit.). These observations go along with the role of mutant Norrin in the Norrie disease.

In sum, Norrin has only been described in the prior art in context of retinal diseases or retinal angiogenesis. Accordingly, Norrin has only be proposed for the treatment of retinal neovascularisation, vascular disorders or vascular abnormalities associated with Norrie disease or other vascular disorders of the retina; see Xu (2004; loc. cit.), Ohlmann (2005; loc. cit.). Treatment of other disorders/diseases and, in particular, diseases associated with an increased TGF-β activity with Norrin has neither been described nor proposed in the art. As mentioned above, the gist of the present invention lies in the unexpected identification of Norrin as TGF-β antagonist/inhibitor and its use in the treatment or prevention of (a) disease(s) associated with an increased TGF-β activity.

This surprising finding is also documented in the appended experimental part. Cell cultures (e.g. mink lung epithelial cells or retinal microvascular endothelial cells) were incubated with TGF-β (in particular TGF-β1) and transgenic mice used in the appended example overexpressed TGF-β1. These cell cultures or transgenic mice can, therefore, serve as exemplary model systems for (a) disease(s) with an increased TGF-β activity.

The experimental results summarized in the following show that Norrin acts as a potent TGF-β antagonist/inhibitor, for example by decreasing TGF-β mediated Luciferase activity in immortalized mink lung epithelial cells (MLEC). MLEC express the reporter gene luciferase under control of a TGF-β1 sensitive PAI (plasminogen activator inhibitor)-1 promoter fragment.

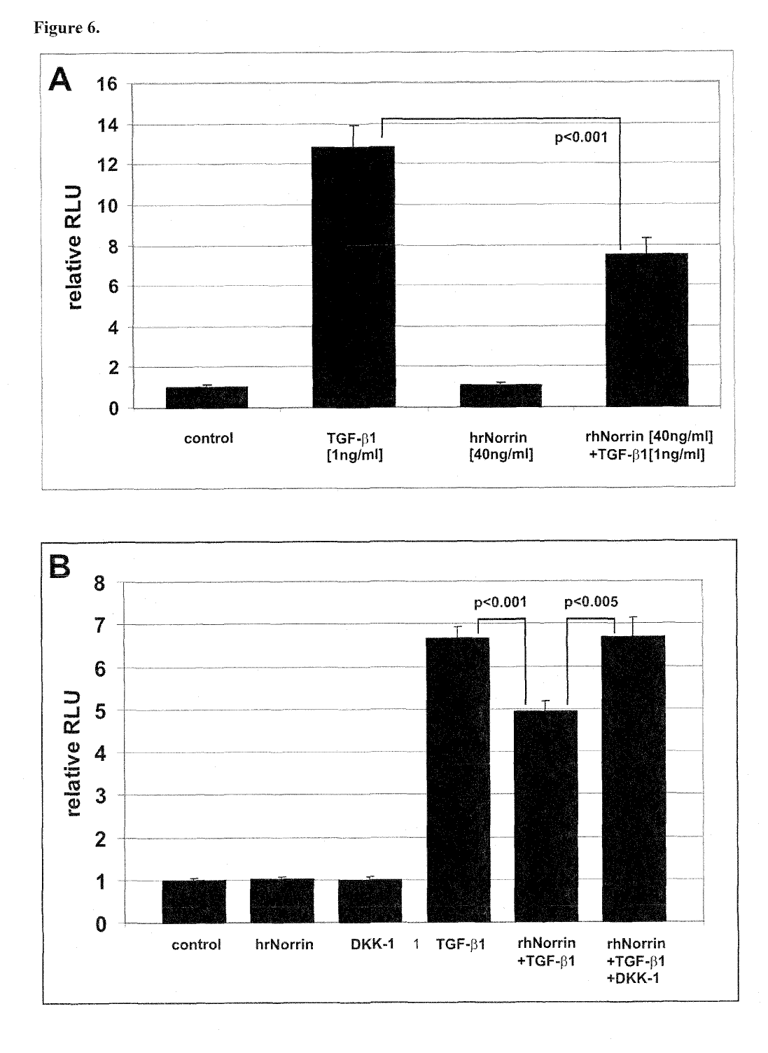

In the presence of TGF-β1 an increase in luciferase activity was observed. Unexpectedly, luciferase activity was highly significantly diminished by more than 40% in the presence of TGF-β1 and human Norrin compared to TGF-β1 alone; see FIG. 6A. Incubation of the cells with TGF-β1, Norrin and Dickkopf (DKK)-1, an antagonist of the frizzled co-receptor low-density lipoprotein receptor-related protein (LRP) type 5 and 6, which is known to be involved in the Wnt signaling pathway, completely restored luciferase activity; see FIG. 6B. Presence of Norrin or DKK-1 alone did not change luciferase activity compared to the control. This clearly shows that Norrin counteracts the activity of TGF-β1.

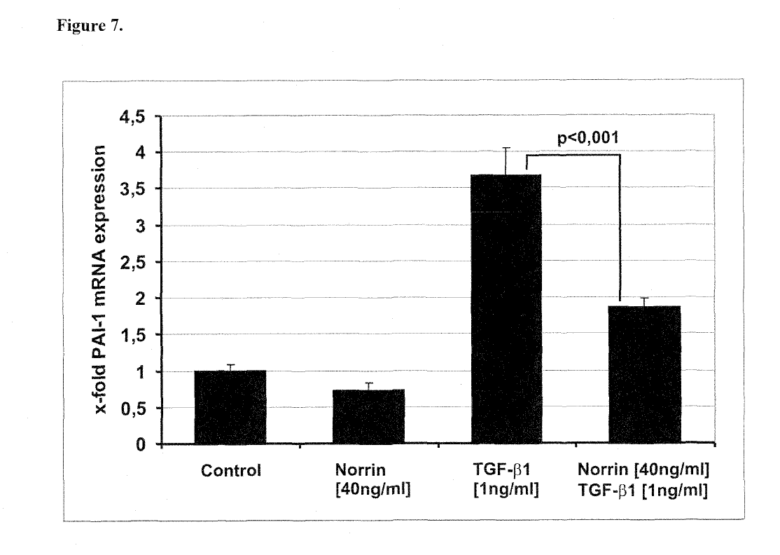

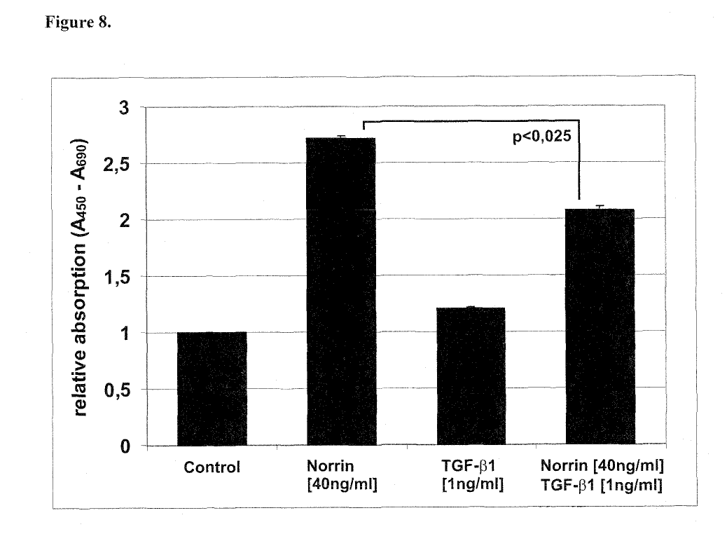

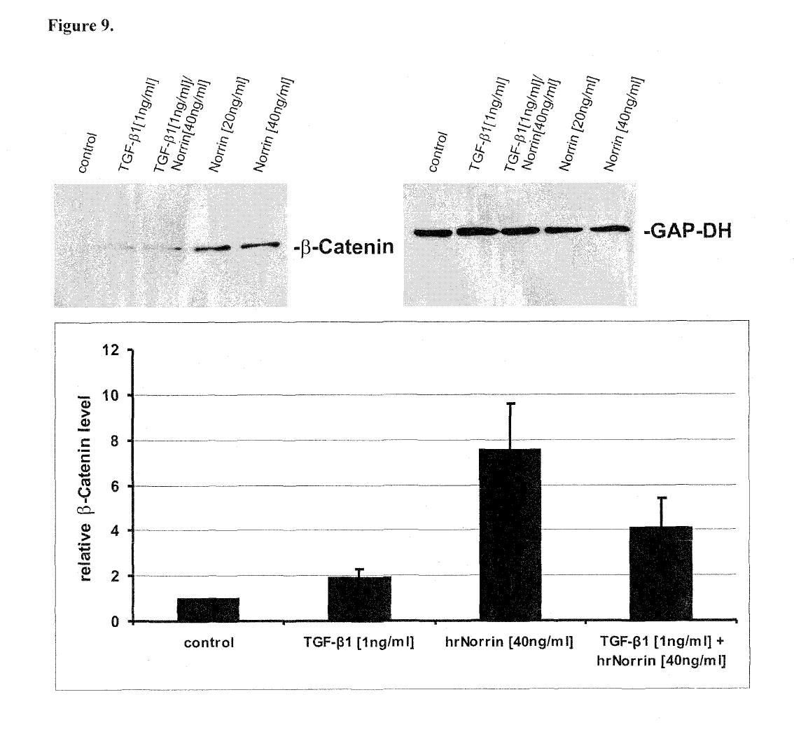

Also the finding that Norrin leads to a marked decrease of about 50% in TGF-β1-induced PAI-1 expression in human dermal microvascular endothelial cells (HDMECs) supports the notion that Norrin acts as suppressor/inhibitor/antagonist of TGF-β1; see FIG. 7. Vice versa, it has been found that TGF-β1 inhibits Norrin-induced proliferation of human retinal microvascular endothelial cells (HRMECs) and thus, may be regarded as suppressor/inhibitor/antagonist of Norrin activity; see FIG. 8.

β-Catenin is known in the art as a central component of the Wnt-signaling pathway. Upon activation of the Wnt-signaling pathway, intracellular β-Catenin levels are increased and β-Catenin is translocated into the nucleus. In the experimental part it is shown that Norrin leads to an about 7.5 fold increase in the level of nuclear β-Catenin over the control which demonstrates that Norrin plays a role in the Wnt-signaling pathway. Also here, presence of TGF-β1 and Norrin markedly reduced β-Catenin levels; see FIG. 9.

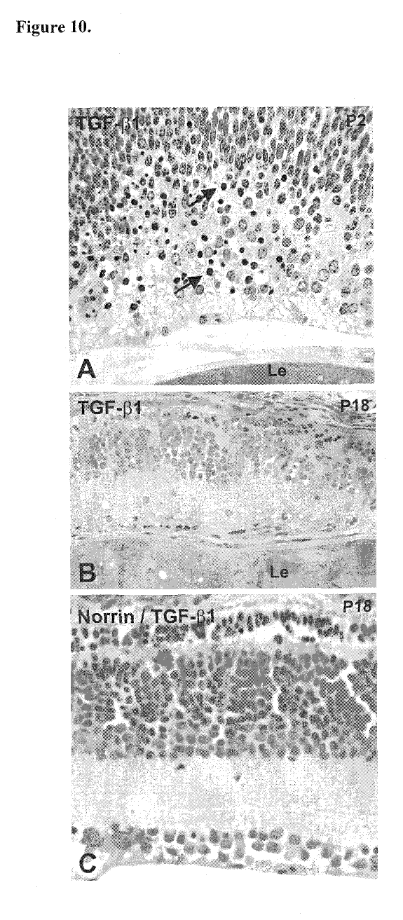

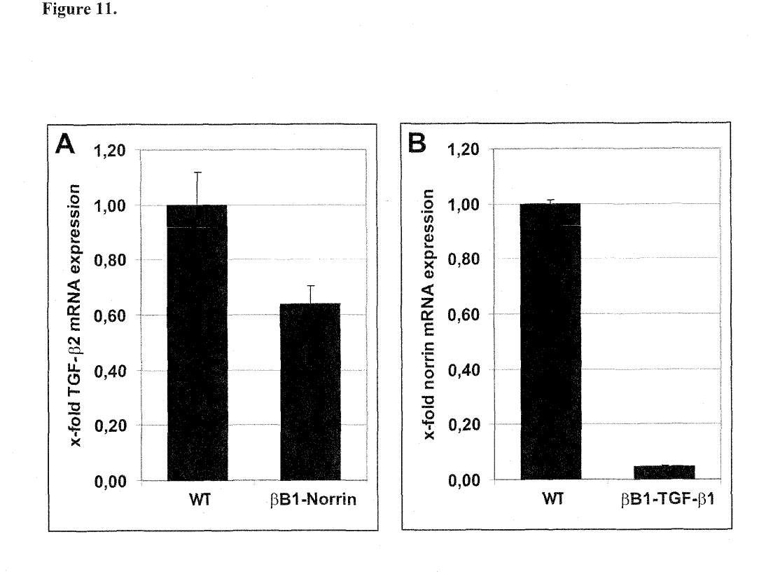

In vivo experiments in transgenic mice confirm the role of Norrin as TGF-β1 antagonist/inhibitor. Crossbreeding of mice expressing Norrin and TGF-β1, respectively, under control of the lens specific βB1-crystallin promoter completely restored the phenotype observed in TGF-β1-transgenic mice; see FIG. 10. Further, it is shown in the appended example that Norrin does not only act as inhibitor/antagonist of TGF-β1 but also of other TGF-isoforms, such as TGF-β2. In the retina of transgenic mice expressing Norrin under control of the lens specific βB1-crystallin promoter a decrease of about 35% in TGF-β2 mRNA expression was observed. Vice versa, in the retina of transgenic mice expressing TGF-β1 under control of the lens specific βB1-crystallin promoter a decrease of about 95% in Norrin mRNA expression was observed; see FIG. 11.

These above described in vitro and in vivo experiments clearly demonstrate that Norrin strongly antagonizes/inhibits the activity of TGF-β, in particular TGF-β1 and TGF-β2 and thus is a potent TGF-β antagonist/inhibitor; see also the appended example. As mentioned above, Norrin or a functional fragment thereof can, due to its activity as TGF-β antagonist/inhibitor, be used in treating or preventing (a) disease(s) associated with an increased TGF-beta activity. A particular advantage of the use of Norrin as described herein is the treatment of (a) disease(s) characterized by an increased TGF-β activity which (has) have not been amenable to treatment with known TGF-β antagonists/inhibitors.

The term “Norrin” used herein refers to a polypeptide with an activity specific for Norrin, and in particular, a TGF-β antagonizing/inhibiting activity, as described herein and shown in the appended example. “Norrin” refers, in particular, to a polypeptide comprising the amino acid sequence shown in SEQ ID NO: 2. Methods for determining the activity of Norrin are described herein below in the context of “functional Norrin”. The Norrin protein may be encoded by a nucleic acid sequence shown in SEQ ID NO: 1. The sequences shown in SEQ ID NO: 1 and SEQ ID NO: 2 refer to the gene encoding the human Norrin protein and the human Norrin protein itself, respectively; see also FIG. 1. However, the present invention is not limited to the use of human Norrin (or a functional fragment thereof) comprising the particular sequences as shown in SEQ ID NOs: 1 and 2, but relates also to the medical use of orthologous or homologous Norrin (or a functional fragment thereof). The terms “orthologous”/“homologous” are described herein below. For example, murine Norrin may be used in context of the present invention. Nucleic acid sequence and amino acid sequence of murine Norrin are shown in SEQ ID NO: 3 and SEQ ID NO: 4, respectively; see also FIG. 2. The respective sequences can also been deduced from public databases. For example, the nucleic acid sequence of human Norrin can be deduced from the NCBI database (accession number NM_{—}000266). Also the nucleic acid sequence of murine Norrin can be deduced from the NCBI database (accession number NM_{—}010883).

In context of the present invention, it is preferred that human Norrin (or Norrin or a functional fragment thereof derived from human Norrin) is used in the treatment of humans suffering from (a) disease(s) associated with an increased TGF-β activity. Correspondingly, murine Norrin (or Norrin or a functional fragment thereof derived from murine Norrin) is preferably used in the treatment of mice suffering from (a) disease(s) associated with an increased TGF-β activity. Accordingly, it is preferred that the Norrin (or functional fragment thereof) to be used in the treatment of a specific organism (e.g. human, mouse or pig) is isolated or derived from a sample from said specific organism (e.g. human, mouse or pig, respectively). Though less preferred, the specific Norrin isolated/derived from a specific organism as described above may also be used in the treatment of closely related organisms; for example, human Norrin may be used in the treatment of a chimpanzee, and vice versa. It is also envisaged that the specific Norrin isolated/derived from a specific organism may also be used in the treatment of distantly related organisms; for example, human Norrin may be used in the treatment of a mouse, and vice versa. Closely related organisms may, in particular, be organisms which form a subgroup of a species, e.g. different races of a species. Also organisms which belong to a different species but can be subgrouped under a common genus can be considered as closely related. Less closely related organisms belong to different genera subgrouped under one family. Distantly related organisms belong to different families. The taxonomic terms “race”, “species”, “genus”, “family” and the like are well known in the art and can easily be derived from standard textbooks. Based on the teaching provided in the present invention are skilled person is therefore easily in the position to identify “closely related” or “distantly related” organisms.

A person skilled in the art is capable of identifying and/or isolating Norrin as defined herein and in particular as defined in sections (a) to (f) of the below-described specific aspect of the present invention or a nucleic acid molecule encoding said Norrin from a specific organism (e.g. human, mouse, pig, guinea pig, rat, and the like) using standard techniques. Again, it is to be understood that Norrin (or a functional fragment thereof) derived from human Norrin, murine Norrin or derived from Norrin isolated from further organisms (e.g. pig, guinea pig, rat, and the like) is to be used in accordance with the present invention, in particular in the treatment or prevention of (a) disease associated with an increased TGFβ-activity.

As used herein the terms “human Norrin”/“Norrin of human origin” refer in particular to (a) protein(s) as found in the human body which can accordingly be isolated from a sample obtained from a human. The term “Norrin (or a functional fragment thereof) derived from human Norrin” refers in particular to “human Norrin”/“Norrin of human origin” which is modified as described herein below (e.g. by way of substitution, deletion and/or insertion of (an) amino acid(s)). Said modified polypeptide may also form part of a fusion protein. The explanations given herein above in respect of “human Norrin”/“Norrin of human origin” apply, mutatis mutandis, to “murine Norrin”/“Norrin of murine origin” and Norrin isolated from other organisms, such as pigs, guinea pigs, rats, and the like.

The use of Norrin (or a functional fragment thereof) as described and defined herein in the treatment of economically, agronomically or scientifically important organisms is envisaged herein. Scientifically or experimentally important organisms include, but are not limited to, mice, rats, rabbits, guinea pigs and pigs. Yet, the treatment of (a) human(s) with Norrin (in particular Norrin of human origin or derived from human Norrin) or a functional fragment thereof is preferred in context of the present invention. Preferably, Norrin to be used in context of the present invention, particular in treating or preventing a disease associated with an increased TGF-β activity, is selected from the group consisting of
(a) a polypeptide comprising an amino acid encoded by a nucleic acid molecule having the nucleic acid sequence as depicted in SEQ ID NO: 1, the nucleic acid sequence comprising nucleic acid residues 4 to 402 in SEQ ID NO: 1 or the nucleic acid sequence comprising nucleic acid residues 73 to 402 in SEQ ID NO: 1;
(b) a polypeptide having an amino acid sequence as depicted in SEQ ID NO:2, an amino acid sequence comprising amino acids 2 to 133 in SEQ ID NO:2 or an amino acid sequence comprising amino acids 25 to 133 in SEQ ID NO:2;
(c) a polypeptide encoded by a nucleic acid molecule encoding a peptide having an amino acid sequence as depicted in SEQ ID NO:2, an amino acid sequence comprising amino acids 2 to 133 in SEQ ID NO:2 or an amino acid sequence comprising amino acids 25 to 133 in SEQ ID NO:2;
(d) a polypeptide comprising an amino acid encoded by a nucleic acid molecule hybridizing under stringent conditions to the complementary strand of nucleic acid molecules as defined in (a) or (c) and encoding a functional Norrin or a functional fragment thereof;
(e) a polypeptide having at least 60% homology to the polypeptide of any one of (a) to (d), whereby said polypeptide is a functional Norrin or a functional fragment thereof; and
(f) a polypeptide comprising an amino acid encoded by a nucleic acid molecule being degenerate as a result of the genetic code to the nucleotide sequence of a nucleic acid molecule as defined in (a), (c) and (d).

The term “functional Norrin” used in context of the present invention refers to a polypeptide having at least 60% homology to a polypeptide as defined in section (a) to (d) of the above-described specific aspect of the present invention which has essentially the same biological activity as a polypeptide having 100% homology to a polypeptide as indicated in section (a) to (d), i.e. a polypeptide being essentially identical to a polypeptide having an amino acid sequence as depicted in SEQ ID NO:2. Methods for determining the activity of (a) polypeptide(s) are well known in the art and may, for example, be deduced from standard text books, such as Bioanalytik (Lottspeich/Zorbas (eds.), 1998, Spektrum Akademischer Verlag). Methods for determining the activity of Norrin of functional Norrin are also described herein below.

It is of note that (functional) Norrin or a functional fragment thereof as described and defined herein may further comprise a heterologous polypeptide, for example, (an) amino acid sequence(s) for identification and/or purification of the recombinant protein (e.g. amino acid sequence from C-MYC, GST protein, FLAG peptide, HIS peptide and the like), an amino acid sequence used as reporter (e.g. green fluorescent protein, yellow fluorescent protein, red fluorescent protein, luciferase, and the like), or antibodies/antibody fragments (like scFV). A person skilled in the art knows that for determination of homology as described herein only a part of a polypeptide to be used herein is to be used, whereby said part is Norrin (or a functional fragment thereof). Also further compounds (e.g. toxins or antibodies or fragments thereof) may be attached to Norrin (or a functional fragment thereof) by standard techniques. These compounds may, in particular, be useful in a medical setting as described herein, wherein Norrin (or a functional fragment thereof) is used. A skilled person is aware of compounds to be used/attached in this context.

In a preferred embodiment, the polypeptide to be used in accordance with the present invention (e.g. Norrin as shown in SEQ ID NO:2 and 4) comprises (a) signal peptide(s), for example a “endogenous” signal peptide present in the “original” Norrin (e.g. as shown in SEQ ID NO. 2 and 4). An exemplary sequence of an “endogenous” signal peptide is depicted in amino acids 1 to 24 in SEQ ID NOs:2 and 4, respectively (corresponding to nucleic acid residues 1 to 72 in SEQ ID NOs: 1 and 3, respectively). Preferably, the polypeptide of the present invention comprises (optionally in addition to the “endogenous” signal peptide(s)) (a) signal peptide(s) of the murine Igκ chain. An exemplary amino acid sequence of a signal peptide of the murine Igκ chain comprises amino acids 1 to 21 in SEQ ID NO:6 (corresponding to nucleic acid residues 1 to 63 in SEQ ID NO: 5). Norrin may comprise (a) further signal peptide(s). The term “signal peptide” is well known in the art and used accordingly herein. Under certain circumstances it may be beneficial that the polypeptide does not comprise a methionine at the N-terminus (e.g. when a “signal peptide” starting with a methionine is to be added at the N-terminus of the polypeptide). Accordingly, also the use of polypeptides lacking an N-terminal methionine is envisaged in the context of the present invention (e.g. a polypeptide having an amino acid sequence comprising amino acids 2 to 133 in SEQ ID NO: 2, amino acids 2 to 131 in SEQ ID NO: 4 or amino acids 2 to 169 in SEQ ID NO: 6 (corresponding to nucleic residues 4 to 402 in SEQ ID NO: 1, 4 to 396 in SEQ ID NO: 3 and 4 to 510 in SEQ ID NO: 5, respectively). A skilled person knows that a polypeptide to be expressed (and e.g. to be secreted as described herein) usually starts with a methionine, whereas this methionine may not be essential for the activity of the polypeptide and can, therefore, be deleted upon expression/secretion. Thus, a polypeptide (i.e. a Norrin or functional fragment thereof) may be used in context of the present invention (e.g. in treating or preventing a disease associated with an increased TGF-beta activity), which lacks a methionine at its N-terminus.

A signal peptide which is, for example, present at the N-terminus of a Norrin (or functional fragment thereof) (e.g. the “original” human Norrin (as shown in SEQ ID NO: 2)) may be removed or replaced by another amino acid sequence, preferably, another signal peptide. As mentioned, a preferred exemplary signal peptide replacing the signal peptide of the “original” human Norrin is the signal peptide of the murine Igκ chain (shown e.g. in amino acids 1 to 21 in SEQ NO: 6). Use of the signal peptide of the murine Igκ chain is particularly preferred in this context since secretion of Norrin (or a functional fragment thereof) can be drastically increased. A Norrin (or a functional fragment thereof) comprising only the endogenous signal peptide (e.g. amino acids 1 to 24 in SEQ ID NOs: 2 and 4) used in overexpression settings (e.g. in cell cultures in order to obtain recombinant Norrin) may be poorly secreted, i.e. the amounts of secreted Norrin may he low or may be barely detectable. In contrast thereto, the secretion of Norrin (or a functional fragment thereof) comprising the signal peptide of the murine Igκ chain (shown e.g. in amino acids 1 to 21 in SEQ NO:6) are preferably increased at least 2-fold, 3-fold, 4-fold, 5-fold, more preferably 6-fold, 7-fold, 8-fold or 9-fold and most preferably at least 10-fold compared to a Norrin (or functional fragment thereof) comprising only the endogenous signal peptide. An increased secretion of Norrin can be determined by methods known in the art. For example, in context of cell cultures producing Norrin, the amount of Norrin secreted into the medium can be determined e.g. by western blots, ELISA and the like. Accordingly, the signal peptide of the murine Igκ chain is of particular advantage in the generation of recombinant Norrin using e.g. eukaryotic cells overexpressing Norrin, since the productivity is increased when compared to the generation of “original” Norrin (e.g. human Norrin as shown in SEQ ID No.2 with the “original” signal peptide). Accordingly, in a preferred embodiment, Norrin as used herein further comprises a signal peptide of the murine Igκ chain. A recombinantly produced Norrin may, for example, also have a different glycosylation pattern when compared to the respective “original” Norrin (e.g. produced in the human or animal body). Replacement of the signal peptide may also lead to a different (subcellular) localisation of Norrin or uptake of Norrin, thus changing and preferably increasing the biological activity of Norrin (or a functional fragment thereof).

A preferred Norrin to be used in accordance with the present invention is shown in SEQ ID NOs: 5 and 6 (nucleotide sequence and amino acid sequence, respectively), wherein said Norrin comprises a signal peptide of the murine Igκ chain as defined and described herein. It is commonly appreciated in the art that a signal peptide is cleaved from the remaining part of a polypeptide during/upon delivery to a particular site, e.g. during/upon secretion. Accordingly, the Norrin (or functional fragment thereof) to be used herein may be devoid of the signal peptide. Such an exemplary Norrin may then comprise an amino acid sequence comprising amino acids 25 to 133 in SEQ ID NO 2 or amino acids 25 to 131 in SEQ ID NO: 4 (corresponding to nucleic acid residues 73 to 402 in SEQ ID NO: 1 and 73 to 396 in SEQ ID NO: 3, respectively).

It is also envisaged herein that Norrin (or a functional fragment thereof) as defined herein, though being of, for example, human, murine or porcine origin (e.g. Norrin isolated from human, mouse or pig as described above), may be modified in order to change certain properties of the polypeptide. For example, such a modified Norrin (or a functional fragment thereof) may, preferably, exhibit increased biological activity as defined herein or increased stability when compared to the “original” Norrin (i.e. the Norrin as produced in a healthy, non-transgenic organism, e.g. human Norrin as defined above). For example, the polypeptide having the amino acid sequence as shown in SEQ ID NO: 2 can be considered as “original” human Norrin, whereas the polypeptide having the amino acid sequence as shown in SEQ ID NO: 4 can be considered as “original” murine Norrin. A person skilled in the art will readily be in the position to identify further “original” Norrin proteins. A “modified” Norrin (or a functional fragment thereof) may have (an) insertion(s), (a) deletion(s) and/or (an) exchange of at least one amino acid.

Methods and assays for determining the activity of “Norrin” are described herein below and in the appended examples. These methods also allow determining whether a polypeptide can be considered as a “functional Norrin”. The activity exhibited by the following exemplary polypeptides can be considered as “reference activity” of a functional Norrin: a polypeptide having an amino acid sequence as depicted in SEQ ID NO:2 (“human Norrin”), a polypeptide having an amino acid sequence comprising amino acids 2 to 133 in SEQ ID NO:2 (“human Norrin” lacking the initial methionine), a polypeptide having an amino acid sequence comprising amino acids 25 to 133 in SEQ ID NO:2 (“human Norrin” lacking the “endogenous” signal peptide), a polypeptide depicted in SEQ ID NO:4 (“murine Norrin”), a polypeptide having an amino acid sequence comprising amino acids 2 to 131 in SEQ ID NO:4 (“murine Norrin” lacking the initial methionine), a polypeptide having an amino acid sequence comprising amino acids 25 to 131 in SEQ ID NO:4 (“murine Norrin” lacking the “endogenous” signal peptide), a polypeptide depicted in SEQ ID NO: 6 (“recombinant human Norrin”), a polypeptide having an amino acid sequence comprising amino acids 2 to 169 in SEQ ID NO:6 (“recombinant human Norrin” lacking the initial methionine) or having an amino acid sequence comprising amino acids 22 to 169 in SEQ ID NO:6 (“recombinant human Norrin” lacking the signal peptide of the murine Igκ chain).

For example, “Norrin” or “functional Norrin” may decrease TGF-β1 mediated Luciferase activity in mink lung epithelial cells (MLECs) by at least about 25%, more preferably by at least about 30%, 35%, 40% or 45% and most preferably by at least about 50% when compared to treatment with TGF-β1 alone (control); see also the appended Example and FIG. 6. “Norrin” or a “functional Norrin” may decrease TGF-β1 mediated PAI-1 mRNA expression by at least about 25%, more preferably by at least 30%, 35%, 40%, 45%, 50%, 55% or 60% and most preferably by at least about 65% when compared to treatment with TGF-β1 alone (control); see also the appended Example and FIG. 7. “Norrin” or a “functional Norrin” may increase the proliferation of HRMEC at least about 1.8 fold, 1.9 fold, 2.0 fold, preferably at least about 2.5 fold, 3.0 fold or 3.5 fold over the control (untreated); see the appended Example and FIG. 8. Further, “Norrin” or a “functional Norrin” may increase nuclear β-Catenin accumulation at least about 7 fold over the control (untreated); see the appended Example and FIG. 9.

Whereas the above relates to in vitro tests, the activity of “Norrin” or “functional Norrin” can also be determined using in vivo tests, for example, by taking advantage of transgenic animals overexpressing “Norrin” or “functional Norrin”. In an exemplary test shown in the appended example, it is demonstrated that overexpression of “Norrin” may rescue the TGF-β1 mediated ocular phenotype of transgenic mice overexpressing TGF-β1. Accordingly, the rescue of a TGF-β1 mediated ocular phenotype (i.e. reverting the phenotype to “normal” or “healthy”) may serve as yet another proof for the activity of “Norrin” or “functional Norrin”; see the appended Example and FIG. 10. Based on his general knowledge and the teaching provided herein, a person skilled in the art is readily in the position to determine whether “Norrin” or “functional” Norrin can revert, for example, a TGF-β1 mediated phenotype. In this context, a skilled person will be aware of various genetic backgrounds of transgenic animals that may be used in these assays, in particular in vivo assays. For example, also transgenic animals deficient in Norrin expression (i.e. having an expression level at least about 20% below normal (healthy, non-transgenic control) and preferably showing no detectable Norrin expression on protein and/or mRNA level) may be used. Overexpression of Norrin may also lead to a reduction in TGF-β2 mRNA expression by at least about 35%; see the appended Example and FIG. 11. Accordingly, “Norrin” or “functional Norrin” may lead to such a reduction in TGF-β2 mRNA expression in appropriate assays.

The above-mentioned assays can be considered as standard methods/assays for determining the (biological) activity of “Norrin” or “functional Norrin”. A person skilled in the art will be aware that (biological) activity as described herein often correlates with the expression level (e.g. protein/mRNA). If not mentioned otherwise, the term “expression” used herein refers to the expression of a nucleic acid molecule encoding a polypeptide/protein, whereas “activity” refers to activity of said polypeptide/protein. The explanations given herein above in respect of determining the activity of “Norrin” and “functional Norrin”, respectively, also apply, mutatis mutandis, to a “functional fragment of Norrin” and to a “functional fragment of a functional Norrin”. In other words, a “functional fragment of Norrin” has essentially the same activity as defined herein above as Norrin and a “functional fragment of a functional Norrin” has, correspondingly, essentially the same activity as defined herein above as a “functional Norrin”. As mentioned, methods/assays for determining the activity of “Norrin”, “functional Norrin”, “functional fragment of Norrin” and “functional fragment of a functional Norrin” are well known in the art and also described herein above.

In one embodiment, the present invention relates to a method for treating or preventing a disease associated with an increased TGF-beta activity comprising the administration of an effective amount of Norrin or a functional fragment thereof as defined herein above to a subject in need of such a treatment or prevention. Preferably, said subject is a human.

The term “treatment of a disease” as used herein is well known in the art. “Treatment of a disease” implies that a disease has been diagnosed in a patient/subject. A patient/subject suspected of suffering from a disease typically shows specific disease symptoms which a skilled person can easily attribute to a specific pathological condition (i.e. diagnose a disease).

As described herein below in detail, a disease to be treated with Norrin (or a functional fragment thereof) is preferably associated with an increase in TGF-beta activity by at least 50%.

“Treatment of a disease” may, for example, lead to a halt in the progression of the disease (e.g. no deterioration of disease symptoms) or a delay in the progression of the disease (in case the halt is of a transient nature only). “Treatment of a disease” may also lead to a partial response (e.g. amelioration of disease symptoms) or complete response (e.g. disappearance of disease symptoms) of the subject/patient suffering from the disease. Such a partial or complete response may be followed by a relapse. It is to be understood that a subject/patient may experience a broad range of responses to a treatment (e.g. the exemplary responses as described herein above).

Treatment of a disease may, inter alia, comprise curative treatment (preferably leading to a complete response and eventually to healing of the disease) and palliative treatment (including symptomatic relief).

Also the term “prevention” as used herein is well known in the art. For example, a patient/subject suspected of being prone to suffer from a disease as defined herein may, in particular, benefit from a prevention of the disease. Said subject/patient may have a susceptibility or predisposition for a disease, including but not limited to hereditary predisposition. Such a predisposition can be determined by standard assays, using, for example, genetic markers. It is to be understood that a disease to be prevented in accordance with the present invention has not been diagnosed or cannot be diagnosed in said patient/subject (for example, said patient/subject does not show any disease symptoms).

In context of the present invention, a disease to be prevented with Norrin (or a functional fragment thereof) is preferably associated with an increase in TGF-beta activity by at least 10% and up to 50%.

A person skilled in the art is, based on his general knowledge and the teaching provided herein, readily capable of identifying (a) disease(s) which (is) are associated with an increased TGF-beta activity, in particular (a) disease(s) associated with an increased TGF-beta 1 and/or TGF-beta 2 activity. As mentioned above, a skilled person will be aware that TGF-beta activity may correlate with the expression level of TGF-beta (e.g. mRNA/protein), i.e. an increase in TGF-beta activity is reflected in an increased expression level of TGF-beta (which can, inter alia, be measured on the protein or mRNA level by standard assays described herein below in detail). Assays for measuring the activity of TGF-beta are well known in the art and are also described herein and used in the appended example.

However, also the use of Norrin (or a functional fragment thereof) in the treatment or prevention of a disease associated with an increased TGF-beta activity is envisaged, wherein the increased TGF-beta activity does not necessarily correlate with an increased TGF-beta level. It is well known in the art that TGF-beta is part of a signaling network with TGF-beta inhibiting or activating factors. For example, a change in these TGF-beta modulating factors can lead to increased activity of TGF-beta while the TGF-beta level is not increased (i.e. the TGF-beta level is essentially the same as in a control sample, e.g. a sample from a healthy organism/subject). A well known example of a protein having a TGF-beta inhibiting activity is Smad7. Mutations in Smad7 may lead to an decreased TGF-beta inhibiting activity and thus lead to increased activity of TGF-beta (while the TGF-beta level may not necessarily be increased). Also the expression of TGF-beta receptors might be increased, allowing binding of more TGF-beta proteins per cell surface area and thus leading to an increased TGF-beta activity. TGF-beta is secreted as latent protein which has to be activated subsequently, for example by the endogenous activator Thrombospondin-1. It is conceivable that an increased activity and/or expression of Thrombospondin-1 may lead to an increased activity of TGF-beta. In this context, it is preferred that the activity of TGF-beta (in particular TGF-beta proteins) is increased by at least about 10%, 20%, 30%, 40%, 50%, 60%, 70%, 80%, 90% or 100% (2 fold concentration/amount), 3 fold, 4 fold, 5 fold, 6 fold, 7 fold, 8 fold, 9 fold or 10 fold in a sample obtained from an organism/patient/subject suspected of suffering from a disease as defined herein compared to a control sample (e.g. a sample obtained from a healthy subject).

The term “increased TGF-beta level” refers to an increased concentration of TGF-beta proteins (in particular an increased concentration of TGF-beta 1 and/or TGF-beta 2 proteins) in (an) organism(s) suffering from such a disease when compared to (a) healthy organism(s) (control) which preferably belongs to the same race, species or is otherwise closely related to the organism suffering from said disease. Typically, the subject/patient/organism suffering from the above-mentioned disease as a whole exhibits an increased concentration of TGF-beta proteins due to, for example, increased TGF-β expression and, optionally, increased secretion of TGF-beta proteins. Yet, some cells, (a) tissue(s) and/or (a) organ(s) (e.g. tumor cells) will exhibit a stronger increase in the concentration of TGF-β or increased secretion of TGF-beta proteins compared to other cells (e.g. non-tumorous cells). For example, an subject/patient/organism suffering from a cancerous disease which is associated with an increased TGF-beta level, will show an increased concentration of TGF-β in or secretion of TGF-β by the tumor(s)/tumorous cell(s). Also (a) tissue(s) and/or cell(s) contacting the tumor(s)/tumorous cell(s) (e.g. non-transformed cells present in the tumor) may show increased concentration of TGF-β due to, for example, an increased uptake of TGF-β, whereas (a) distant cell(s), tissue(s) and/or organ(s) typically show a less pronounced increased concentration of TGF-β or no increase at all. Accordingly, a sample obtained from a patient/subject/organism suspected of suffering from a disease associated with an increased TGF-beta level is used herein, wherein the sample is assumed to comprise cells having an increased TGF-beta level.

As mentioned, an increased TGF-beta level is reflected in an increased concentration/amount of (functional) TGF-beta proteins (and optionally, unspliced/partially spliced/spliced mRNA) in a sample obtained from an organism suspected of suffering from (a) disease associated with such an increased TGF-beta level when compared to a healthy (control) organism. Biological samples to be assessed are described herein below in more detail. The increased concentration/amount of TGF-beta proteins may, for example, be due to an increased expression of the corresponding gene(s) encoding the TGF-beta protein(s) and/or increased stability of TGF-beta protein(s). A person skilled in the art is easily in the position to determine the concentration/amount of TGF-beta proteins in a sample and deduce whether the concentration/amount is increased when compared to a control sample. For example, the concentration/amount of TGF-beta proteins may be increased by at least about 10%, 20%, 30%, 40%, 50%, 60%, 70%, 80%, 90% or 100% (2 fold concentration/amount), 3 fold, 4 fold, 5 fold, 6 fold, 7 fold, 8 fold, 9 fold or 10 fold compared to a control sample.

In the context of “preventing a disease associated with increased TGF-beta activity” as described herein the concentration/amount of TGF-beta proteins may, in particular, be increased by at least about 10%, 20%, 30%, 40% and up to 50% compared to a control sample.

In context of “treating a disease associated with increased TGF-beta activity” the concentration/amount of TGF-beta proteins may, in particular, be increased by at least 50%, preferably by at least about, 60%, 70%, 80%, 90% or 100% (2 fold) compared to a control sample. In particular, the concentration/amount of TGF-beta proteins may, in this context, be at least about 3 fold, 4 fold, 5 fold, 6 fold, 7 fold or 8 fold compared to a control sample.

A skilled person is also aware of standard methods to be used in determining the amount/concentration of TGF-β-proteins in a sample or may deduce corresponding methods from standard textbooks (e.g. Sambrook, 2001). For example, concentration/amount of TGF-beta proteins in biological fluids or cell lysates can be determined by enzyme linked-immunosorbent assay (ELISA). Alternatively, Western Blot analysis or immunohistochemical staining can be performed.

In samples obtained from cell(s)/cell culture(s) transfected with appropriate constructs or obtained from transgenic animals or cell cultures derived from transgenic animals, wherein the transgenic animal(s) suffer(s) from a disease associated with an increased TGF-β level, the concentration/amount of (bioactive/functional) TGF-β protein can be determined by bioassays, if, for example, a TGF-β-inducible promoter is fused to a reporter gene. Apparently, increased expression of the reporter gene/activity of the reporter gene product will reflect an increased TGF-β level, in particular an increased concentration/amount of (functional) TGF-beta protein. An exemplary bioassay based on mink lung epithelial cells (MLEC) stably transfected with the reporter gene luciferase under control of the TGF-β1 sensitive/inducible PAI-1 (plasminogen activator inhibitor-1) promoter fragment is also described in the appended example. As demonstrated in the example, an increase in the amount/concentration of TGF-β1 protein in a sample leads to a marked increase in luciferase activity; see FIG. 6. Alternatively, the effect of TGF-proteins on the expression of (a) reporter gene(s) may be evaluated by determining the amount/concentration of the gene product of the reporter gene(s) (e.g. protein or spliced, unspliced or partially spliced mRNA). In the experimental part, it has been demonstrated that also a bioassay may be used in determining the amount of bioactive TGF-β protein, wherein the mRNA as reporter gene product is used, wherein the reporter gene is under control of a TGF-β-inducible promoter. It is shown in the appended example that an increase in the amount/concentration of TGF-β1 protein in a sample leads to a marked increase (at least about 3.5 fold) in the mRNA concentration/amount of the reporter gene PAI-1 see FIG. 7. Further methods to be used in the assessment of mRNA expression of a reporter gene are within the scope of a skilled person and also described herein below.

As mentioned, an increased TGF-β level and, accordingly, an increased concentration/amount of TGF-beta proteins in a sample may be reflected in an increased expression of the corresponding gene(s) encoding the TGF-beta protein(s). Therefore, a quantitative assessment of the gene product (e.g. protein or spliced, unspliced or partially spliced mRNA) can be performed in order to evaluate increased expression of the corresponding gene(s) encoding the TGF-beta protein(s). Also here, a person skilled in the art is aware of standard methods to be used in this context or may deduce these methods from standard textbooks (e.g. Sambrook, 2001, loc. cit.). For example, quantitative data on the respective concentration/amounts of mRNA from TGF-β can be obtained by Northern Blot, Real Time PCR and the like. Preferably, the concentration/amount of the gene product (e.g. the herein above described TGF-β mRNA or TGF-β protein) may be increased by at least about 10%, 20%, 30%, 40%, 50%, 60%, 70%, 80%, 90%,100% (2 fold concentration/amount), 3 fold, 4 fold, 5 fold, 6 fold, 7 fold, 8 fold, 9 fold or 10 fold compared to a control sample. It is preferred herein that TGF-β proteins are (biologically) active or functional. Methods for determining the activity of TGF-β are described herein above and shown in the appended example. Since the TGF-β proteins are preferably (biologically) active/functional (wherein it is preferred that at least 70%, 75%, 80%, 85%, 90%, 95%, 96%, 97%, 98% and most preferably, at least 99% of TGF-β proteins of a sample a (biologically) active/functional), an increased concentration/amount of TGF-beta proteins in a sample reflects a higher (biological) acitivity of TGF-beta proteins, and vice versa.

As mentioned, a person skilled in the art is aware of standard methods to be used for determining or quantitating expression of a nucleic acid molecule encoding, for example, the TGF-beta protein(s) or Norrin (or a functional fragment thereof) as defined herein. For example, the expression can be determined on the protein level by taking advantage of immunoagglutination, immunoprecipitation (e.g. immunodiffusion, immunelectrophoresis, immune fixation), western blotting techniques (e.g. (in situ) immuno histochemistry, (in situ) immuno cytochemistry, affinitychromatography, enzyme immunoassays), and the like. Amounts of purified polypeptide in solution can be determined by physical methods, e.g. photometry. Methods of quantifying a particular polypeptide in a mixture rely on specific binding, e.g of antibodies. Specific detection and quantitation methods exploiting the specificity of antibodies comprise for example immunohistochemistry (in situ). Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction.

Expression can also be determined on the nucleic acid level (e.g. if the gene product/product of the coding nucleic acid sequence is an unspliced/partially spliced/spliced mRNA) by taking advantage of Northern blotting techniques or PCR techniques, like in-situ PCR or Real time PCR. Quantitative determination of mRNA can be performed by taking advantage of northern blotting techniques, hybridization on microarrays or DNA chips equipped with one or more probes or probe sets specific for mRNA transcripts or PCR techniques referred to above, like, for example, quantitative PCR techniques, such as Real time PCR.

These and other suitable methods for detection and/or determination of the concentration/amount of (specific) mRNA or protein(s)/polypeptide(s) are well known in the art and are, for example, described in Sambrook and Russell (2001, loc. cit.).

A skilled person is capable of determining the amount of mRNA or polypeptides/proteins, in particular the gene products described herein above, by taking advantage of a correlation, preferably a linear correlation, between the intensity of a detection signal and the amount of, for example, the mRNA or polypeptides/proteins to be determined.

It is of note that (a) disease(s) associated with an increased TGF-β level may, in particular, be associated with an increased level of (a) TGF-β isoform(s), i.e. “TGF-β” refers in this context to (a) TGF-β isoform(s). The term “TGF-β isoform” means in context of the present invention a protein or functional fragment thereof having TGF-β activity as described and defined herein above. In particular, the term “TGF-β isoform” refers to TGF-β1 and TGF-β2 which are well known in the art. Exemplary diseases which are known to be associated with an increased TGF-β level are described herein below in more detail. These diseases may be associated with an increased level of one TGF-β isoform (e.g. TGF-β1 or TGF-β2) or an increased level of more than one TGF-β isoform (e.g. TGF-β1 and TGF-β2, and optionally (a) further TGF-β isoform(s)). It is to be understood that the TGF-β isoform(s) may exhibit a different increase rate. For example, the amount/concentration of TGF-β1 may be 9 fold when compared to a control sample and/or the amount/concentration of TGF-β2 may be 7 fold when compared to a control sample. It is well known in the art that a simultaneous increase in the amount/concentration of different TGF-β isoforms (e.g. TGFβ-1 and TGF-β2) is rarely observed in a sample since TGF-β1 and TGF-β2 are expressed in a tissue specific manner. In pathological conditions, an increased level of TGF-β can, for example, be found in the CNS or in the eye. Generally, all explanations and definitions given herein above and below in respect of “TGF-β” or “increased TGF-β level” apply, mutatis mutandis, in respect of TGF-β isoforms, in particular TGF-β1 or TGF-β2, and vice versa.

The terms “TGF-β” and “TGF-beta” and further grammatical variants thereof can be used interchangeably herein.

It is envisaged in context of the present invention that the “(biological) sample(s)” to be used in the assessment of TGF-β level may be (a) biological or medical sample(s), like, e.g. (a) sample(s) comprising cell(s) or tissue(s). For example, such (a) sample(s) may comprise(s) biological material of biopsies. The meaning of “biopsies” is known in the art. For instance, biopsies comprise cell(s) or tissue(s) taken, e. g. by the attending physician, from a patient/subject/organism suffering or being suspected of suffering from the herein defined fibrotic or proliferative diseases, in particular cancerous diseases (e.g. chronic pancreatic inflammation, pancreatic fibrosis, cystic fibrosis, fibrosis of the lung, malignant melanoma, pancreas carcinoma and the like). Further, non-limiting examples of fibrotic or proliferative diseases are given herein below. It is preferred that (a) biological sample(s) to be used is (are) obtained from a patient/subject/organism suffering from the above mentioned disease(s), wherein the disease(s) is suspected of being associated with an increased TGF-β activity (or TGF-β level).

(A) non-limiting example(s) of the biological/medical/pathological sample(s) to be analysed in context of the present invention is (are) (a) sample(s) which is (are) or is (are) derived from blood, plasma, white blood cells, urine, semen, sputum, cerebrospinal fluid, aqueous humour, vitreous body, lymph or lymphatic tissues or cells, muscle cells, heart cells, nerve cells, cells from spinal cord, brain cells, liver cells, kidney cells, cells from the intestinal tract, colon cells skin, bone, bone marrow, placenta, amniotic fluid, hair, hair and/or follicles, stem cells (embryonal, neuronal, and/or others) or primary or immortalized cell lines (lymphocytes, macrophages, or cell lines). In case of fibrotic diseases described herein, the biological/medical/pathological sample(s) is (are) preferably obtained from fibrotic tissue(s) and/or fibrotic cell(s). In case of proliferative diseases, in particular cancerous diseases, the biological/medical/pathological sample(s) is (are) obtained from cancerous/tumorous tissue(s) and/or cancer/tumor cell(s). As mentioned, the biological, medical or pathological sample as defined herein may also be or be derived from biopsies, in particular biopsies comprising fibrotic/cancerous tissue(s). The biological/medical/pathological samples, like body fluids, isolated body tissue samples and the like, preferably comprise cells or cell debris to be analyzed.

The following relates to diseases known or suspected of being associated with an increased TGF-β activity, and in particular with an increased TGF-β level. The prior art literature recited herein below documents in particular an increased TGF-β level in specific diseases.

As mentioned above, Norrin or a functional fragment thereof as defined herein above has surprisingly been found in the present invention to be particularly useful in the treatment or prevention of (a) disease(s) associated with an increased TGF-beta activity, like (a) fibrotic disease(s) or (a) proliferative disease(s). The meaning of the terms “fibrotic disease” and “proliferative disease” is well known in the art and may be deduced from review articles (see Wynn (2008) J Pathol 214(2), 199-210) or standard textbooks like Harrison's Principles of Internal Medicine, 17th Edition McGraw-Hill Professional (Mar. 6, 2008), Roche Lexikon Medizin, Urban & Fischer, 5^{th} edition, Elsevier (2006).

Non-limiting exemplary fibrotic diseases to be treated or prevented in accordance with the present invention are chronic pancreatitis and pancreatic fibrosis (Talukdar (2006), Pancreatology 6, 440-449; Talukdar (2008), J Gastroenterol Hepatol 23, 34-41), fibrosis of the conjunctiva (Cordeiro (1999), Invest Ophtalmol Vis Sci 40, 1975-1982; Cordeiro (2003), Clin Sci (Lond) 104, 181-7; Picht (2001), Graefes Arch Clin Exp Ophtalmol 239, 199-207), cystic fibrosis, injection fibrosis, endomyocardial fibrosis, mediastinal fibrosis, myleofibrosis, retroperitoneal fibrosis, nephrogenic systemic fibrosis, diabetic nephropathy (Kanwar (2008), Exp Biol Med (Maywood) 233, 4-11), post-vasectomy pain syndrome, rheumatoid arthritis, fibrosis of the lung, liver fibrosis (Friedman (2008), Gastroenterology 134, 1655-69), cirrhosis, dermal keloids and excessive scarring (Jagadeesan (2007) Int J Surg 5, 278-85), scleroderma, fibrosis of the kidneys, glomerulosclerosis of the kidneys, remodeling during myocardial infarct healing with shortening and thickening of the infarcted segment (Bujak (2007), Cardiovasc Res 74, 184-95; Okada (2005), Circulation 111, 2430-7), failure after filtrating glaucoma surgery (Cordeiro (1999), loc. cit.; Cordeiro (2003), loc. cit.), glaucoma and cardiomyopathy with increased TGF-beta level (Li (1997), Circulation 96, 874-81). Exemplary fibrotic lung diseases (fibrosis of the lung) are Acute Respiratory Distress Syndrome (ARDS), chronic obstructive pulmonary disease (COPD), idiopathic pulmonary fibrosis, asbestosis, progressive massive fibrosis, drug-induced lung fibrosis (Cutroneo (2007), J Cell Physiol 211, 585-589) and fibrosis resulting from pulmonary hypertension and asthma.

Alternatively, Norrin or a functional fragment thereof, may be used in the treatment or prevention of (a) proliferative disease(s), in particular (a) cancerous disease(s). The meaning of the term “cancerous disease” is well known in the art and may be deduced from review articles (see Wynn (2008; loc. cit) or standard textbooks like Harrison's Principles of Internal Medicine (2008; loc. cit.) or Roche Lexikon Medizin (2006; loc. cit.).

It is known in the art that many cancerous diseases or tumorous diseases show an increased secretion/expression of TGF-β1 or TGF-β2 when compared to the corresponding normal (healthy) tissue. The association of TGF-βs (TGF-β isoforms as described above) with cancer is strongest in the most advanced stages of tumor progression. The degree of TGF-β overexpression correlates with the severity of the tumor grade. The large amounts of TGF-βs that are secreted by malignant cells act on nontransformed (non-tumorous/normal/healthy) cells present in the tumor mass as well as distal cells in the host in order to suppress antitumor immune responses, thus creating an environment of immune tolerance, augmenting angiogenesis, invasion and metastasis, and increasing tumor extracellular matrix deposition. A critical factor that contributes to metastasis and poor prognosis is TGF-β induced epithelial to mesenchymal transition; see Akhurst (2001), Trends Cell Biol 11, S44-51; Bierie (2006), Nat Rev Cancer 6, 506-20; Derynck (2001), Nat Genet 29, 117-29; Derynck (1987), Cancer Res 47, 707-12; Jakowlew (2006), Cancer Metastasis Rev 25, 435-57; Teicher (2001), Cancer Metastasis Rev 20, 133-43; Teicher (2007), Clin Cancer Res 13, 6247-51.

Cancerous diseases/tumorous diseases/tumors in which an increased TGF-β secretion and a negative influence of TGF-β on prognosis has been found include but are not limited to malignant melanoma (Krasagakis (1998), Br J Cancer 77, 1492-4; Reed (1994), Am J Pathol 145, 97-104), malignant glioma (Jachimczak (1996), Int J Cancer 65, 332-7; Jennings (1991), Int J Cancer 49, 129-39; Kjellman (2000), Int J Cancer 89, 251-8), malignant tumors of the central nervous system (CNS), pancreas carcinoma (Friess (1993), Gastroenterology 105, 1846-56; von Bernstorff (2001), Clin Cancer Res 7, 925s-932s), colorectal carcinoma (Friedman (1995), Cancer Epidemiol Biomarkers Prey 4, 549-54; Tsamandas (2004), Strahlenther Onkol 180, 201-8; Tsushima (1996), Gastroenterology 110, 375-82), non-small cell lung cancer (Hasegawa (2001), Cancer 91, 964-71), prostate carcinoma (Steiner (1994), Endocrinology 135, 2240-7; Truong (1993), Hum Pathol 24, 4-9; Wikstrom (2001), Microsc Res Tech 52, 411-9), hepatocellular carcinoma (Matsuzaki (2000), Cancer Res 60, 1394-402), hematological malignancies (Kyrtsonis (1998), Med Oncol 15, 124-8), renal cell carcinoma (Ananth (1999), Cancer Res 59, 2210-6; Kominsky (2007), J Bone Miner Res 22, 37-44; Weber (2007), Cancer Metastasis Res 26, 691-704), cutaneous squamous cell carcinomas (Johansson (2000), J Cell Sci 113 Pt2, 227-35; Leivonen (2006), Oncogene 25, 2588-600), esophageal carcinoma (Sun (2007), World J Gastroenterol 13, 5267-72) and breast carcinoma (Nicolini (2006), Cytokine Growth Factor Rev 17, 325-37).

Yet, also further fibrotic or proliferative diseases may be treated or prevented with the means and methods provided herein. Such proliferative disorders do not only comprise primary cancers/tumors, but also secondary tumors (i.e. tumors that develop due to metastatic events).

The terms “Norrin”, “TGF-β” and “TGF-β isoform(s)” (like TGF-β1 and TGF-β2) have been described herein above in detail. The terms “TGF-β”, “TGF-β isoform(s)”, “TGF-β1”, “TGF-β2” and the like can be used interchangeably with the terms “TGF-beta”, “TGF-beta isoform(s)”, “TGF-beta1”, “TGF-beta2” and the like. Nucleic acid sequences and amino acid sequences of human or murine Norrin are shown in SEQ ID NOs 1 and 2 or 3 and 4, respectively. Also amino acid sequences and nucleic acid sequences of human or murine TGF-β isoform(s), in particular TGF-β1 and TGF-β2, are depicted in SEQ ID NOs 7 to 10. Information on TGF-β1 and TGF-β2 (in particular of the corresponding nucleic acid and amino acid sequences) can also be retrieved from http://www.ncbi.nlm.nih.gov/sites/entrez?db=omim, OMIM®—Online Mendelian Inheritance in Man®; TGFbeta1: *190180, bzw. http://www.ncbi.nlm.nih.gov/entrez/dispomim.cgi?id=190180; TGF-beta2: *190220 oder http://www.ncbi.nlm.nih.gov/entrez/dispomim.cgi?id=190220 The corresponding sequences of human and murine Norrin are provided in the appended FIGS. 1 and 2.

The meanings of the term “polypeptide” and “nucleic acid sequence(s)/molecule(s)” are well known in the art and are used accordingly in context of the present invention. For example, “nucleic acid sequence(s)/molecule(s)” as used herein refer(s) to all forms of naturally occurring or recombinantly generated types of nucleic acids and/or nucleic acid sequences/molecules as well as to chemically synthesized nucleic acid sequences/molecules. This term also encompasses nucleic acid analogs and nucleic acid derivatives such as e. g. locked DNA, PNA, oligonucleotide thiophosphates and substituted ribo-oligonucleotides. Furthermore, the term “nucleic acid sequence(s)/molecules(s)” also refers to any molecule that comprises nucleotides or nucleotide analogs.

Preferably, the term “nucleic acid sequence(s)/molecule(s)” refers to deoxyribonucleic acid (DNA) and ribonucleic acid (RNA). The “nucleic acid sequence(s)/molecule(s)” may be made by synthetic chemical methodology known to one of ordinary skill in the art, or by the use of recombinant technology, or may be isolated from natural sources, or by a combination thereof. The DNA and RNA may optionally comprise unnatural nucleotides and may be single or double stranded. “Nucleic acid sequence(s)/molecule(s)” also refers to sense and anti-sense DNA and RNA, that is, a nucleotide sequence which is complementary to a specific sequence of nucleotides in DNA and/or RNA.

Furthermore, the term “nucleic acid sequence(s)/molecule(s)” may refer to DNA or RNA or hybrids thereof or any modification thereof that is known in the state of the art (see, e.g., U.S. Pat. No. 5,525,711, U.S. Pat. No. 4,711,955, U.S. Pat. No. 5,792,608 or EP 302175 for examples of modifications). The nucleic acid molecule(s) may be single- or double-stranded, linear or circular, natural or synthetic, and without any size limitation. For instance, the nucleic acid molecule(s) may be genomic DNA, cDNA, mRNA, antisense RNA, ribozymal or a DNA encoding such RNAs or chimeroplasts (Colestrauss, Science (1996), 1386-1389). Said nucleic acid molecule(s) may be in the form of a plasmid or of viral DNA or RNA. “Nucleic acid sequence(s)/molecule(s)” may also refer to (an) oligonucleotide(s), wherein any of the state of the art modifications such as phosphothioates or peptide nucleic acids (PNA) are included.

The nucleic acid sequence of Norrin or TGF-β/TGF-β isoform(s) of other species than the herein provided human and murine sequences for Norrin or TGF-β/TGF-β isoform(s) can be identified by the skilled person using methods known in the art, e.g. by using hybridization assays or by using alignments, either manually or by using computer programs such as those mentioned herein below in connection with the definition of the term “hybridization” and degrees of homology. In one embodiment, the nucleic acid sequence encoding for orthologs of human Norrin or TGF-β/TGF-β isoform(s) is at least 40% homologous to the nucleic acid sequence as shown in SEQ ID NO. 1, 7 and 9 respectively. More preferably, the nucleic acid sequence encoding for orthologs of human Norrin or TGF-β/TGF-β isoform(s) is at least 45%, 50%, 55%, 60%, 65%, 70%, 75%, 80%, 85%, 90%, 91%, 92%, 93%, 94%, 95%, 96%, 97% or 98% homologous to the nucleic acid sequence as shown in SEQ ID NOs. 1, 7 and 9, respectively, wherein the higher values are preferred. Most preferably, the nucleic acid sequence encoding for orthologs of human Norrin or TGF-β/TGF-β isoform(s) is at least 99% homologous to the nucleic acid sequence as shown in SEQ ID NOs 1, 7 and 9, respectively. The term “orthologous protein” or “orthologous gene” as used herein refers to proteins and genes, respectively, in different species that are similar to each other because they originated from a common ancestor.

The same definitions given herein in respect of orthologs/homologs of human Norrin (including, for example, recombinant human Norrin as shown in SEQ ID NO: 5) and human TGF-β/TGF-β isoform(s) apply, mutatis mutandis, to orthologs/homologs of murine Norrin and murine TGF-β/TGF-β isoform(s), in particular the nucleic acid sequence of murine Norrin as shown in SEQ ID NO: 3. The definitions and explanations also apply, mutatis mutandis, to Norrin and TGF-β/TGF-β isoform(s) isolated/derived from further sources, like the herein described animal sources such as pigs or guinea pigs and the like.

Hybridization assays for the characterization of orthologs of known nucleic acid sequences are well known in the art; see e.g. Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989). The term “hybridization” or “hybridizes” as used herein may relate to hybridizations under stringent or non-stringent conditions. If not further specified, the conditions are preferably non-stringent. Said hybridization conditions may be established according to conventional protocols described, e.g., in Sambrook (2001) loc. cit.; Ausubel (1989) loc. cit., or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as, for example, the highly stringent hybridization conditions of 0.1×SSC, 0.1% SDS at 65° C. or 2×SSC, 60° C., 0.1% SDS. Low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions.

In accordance with the present invention, the terms “homology” or “percent homology” or “identical” or “percent identity” or “percentage identity” or “sequence identity” in the context of two or more nucleic acid sequences refers to two or more sequences or subsequences that are the same, or that have a specified percentage of nucleotides that are the same (preferably at least 40% identity, more preferably at least 45%, 50%, 55%, 60%, 65%, 70%, 75%, 80%, 85%, 90%, 91%, 92%, 93%, 94%, 95%, 96%, 97% or 98% identity, most preferably at least 99% identity), when compared and aligned for maximum correspondence over a window of comparison, or over a designated region as measured using a sequence comparison algorithm as known in the art, or by manual alignment and visual inspection. Sequences having, for example, 75% to 90% or greater sequence identity may be considered to be substantially identical. Such a definition also applies to the complement of a test sequence. Preferably the described identity exists over a region that is at least about 15 to 25 nucleotides in length, more preferably, over a region that is at least about 50 to 100 nucleotides in length and most preferably, over a region that is at least about 800 to 1200 nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. The BLOSUM62 scoring matrix (Henikoff (1989) PNAS 89:10915) uses alignments (B) of 50, expectation (E) of 10, M=5, N=4, and a comparison of both strands.

In order to determine whether an nucleotide residue in a nucleic acid sequence corresponds to a certain position in the nucleotide sequence of e.g. SEQ ID NOs: 1, 7 and 9, respectively, the skilled person can use means and methods well-known in the art, e.g., alignments, either manually or by using computer programs such as those mentioned herein. For example, BLAST 2.0, which stands for Basic Local Alignment Search Tool BLAST (Altschul (1997), loc. cit.; Altschul (1993), loc. cit.; Altschul (1990), loc. cit.), can be used to search for local sequence alignments. BLAST, as discussed above, produces alignments of nucleotide sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cut-off score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules. Another example for a program capable of generating sequence alignments is the CLUSTALW computer program (Thompson (1994) Nucl. Acids Res. 2:4673-4680) or FASTDB (Brutlag (1990) Comp. App. Biosci. 6:237-245), as known in the art.

The explanations and definitions given herein above in respect of “homology of nucleic acid sequences” apply, mutatis mutandis, to “amino acid sequences”, in particular an amino acid sequence as depicted in SEQ ID NO: 2 (“human Norrin”), SEQ ID NO: 4 (“murine Norrin”), SEQ ID NO: 6 (“recombinant human Norrin”), SEQ ID NO: 8 (human TGF-beta 1) and SEQ ID NO: 10 (human TGF-beta 2). In one embodiment, the polypeptide to be used in accordance with the present invention has at least 60% homology to the polypeptide having the amino acid sequence as depicted in SEQ ID NOs: 2, 4, 6, 8 and 10, respectively. More preferably, the polypeptide has at least 45%, 50%, 55%, 60%, 65%, 70%, 75%, 80%, 85%, 90%, 91%, 92%, 93%, 94%, 95%, 96%, 97% or 98% homology to the polypeptide having the amino acid sequence as depicted in SEQ ID NO: 2, wherein the higher values are preferred. Most preferably, the polypeptide has at least 99% homology to the polypeptide having the amino acid sequence as depicted in SEQ ID NOs: 2, 4, 6, 8 and 10, respectively.

The terms “complement”, “reverse complement” and “reverse sequence” referred to herein are described in the following example: For sequence 5′AGTGAAGT3′, the complement is 3′TCACTTCA5′, the reverse complement is 3′ACTTCACT5′ and the reverse sequence is 5′TGAAGTGA3′.

In the following, pharmaceutical compositions and Norrin (or a functional fragment thereof) to be prepared and used in accordance with the present invention, in particular in gene therapy, are described.

The pharmaceutical composition will be formulated and dosed in a fashion consistent with good medical practice, taking into account the clinical condition of the individual patient, the site of delivery of the pharmaceutical composition, the method of administration, the scheduling of administration, and other factors known to practitioners. The “effective amount” of the pharmaceutical composition for purposes herein is thus determined by such considerations. The term “effective amount” as used herein refers in particular to a tolerable dose of Norrin or a functional fragment thereof as defined herein which is high enough to cause, for example, depletion of pathologic cells, tumor elimination, tumor shrinkage or stabilization of a disease associated with an increased TGF-β level without or essentially without major toxic effects. Such effective and non-toxic doses may be determined e.g. by dose escalation studies described in the art and should be below the dose inducing severe adverse side events (dose limiting toxicity, DLT).

The skilled person knows that the effective amount of pharmaceutical composition administered to an individual will, inter alia, depend on the nature of the compound. For example, if said compound is a (poly)peptide or protein the total pharmaceutically effective amount of pharmaceutical composition administered parenterally per dose will be in the range of about 1 μg protein/kg/day to 10 mg protein/kg/day of patient body weight, although, as noted above, this will be subject to therapeutic discretion. More preferably, this dose is at least 0.01 mg protein/kg/day, and most preferably for humans between about 0.01 and 1 mg protein/kg/day. If given continuously, the pharmaceutical composition is typically administered at a dose rate of about 1 μg/kg/hour to about 50 μg/kg/hour, either by 1-4 injections per day or by continuous subcutaneous infusions, for example, using a mini-pump. An intravenous bag solution may also be employed. The length of treatment needed to observe changes and the interval following treatment for responses to occur appears to vary depending on the desired effect. The particular amounts may be determined by conventional tests which are well known to the person skilled in the art.

Pharmaceutical compositions of the invention may be administered orally, rectally, parenterally, intracisternally, intravaginally, intraperitoneally, topically (as by powders, ointments, drops or transdermal patch), bucally, intravitreally (e.g. injected into the vitreous body), intracamerally (e.g. injected into the anterior chamber) or as an oral or nasal spray.

Pharmaceutical compositions of the invention preferably comprise a pharmaceutically acceptable carrier. By “pharmaceutically acceptable carrier” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

The pharmaceutical composition is also suitably administered by sustained release systems. Suitable examples of sustained-release compositions include semi-permeable polymer matrices in the form of shaped articles, e.g., films, or mirocapsules. Sustained-release matrices include polylactides (U.S. Pat. No. 3,773,919, EP 58,481), copolymers of L-glutamic acid and gamma-ethyl-L-glutamate (Sidman, U. et al., Biopolymers 22:547-556 (1983)), poly(2-hydroxyethyl methacrylate) (R. Langer et al., J. Biomed. Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Sustained release pharmaceutical composition also include liposomally entrapped compound. Liposomes containing the pharmaceutical composition are prepared by methods known per se: DE 3,218,121; Epstein et al., Proc. Natl. Acad. Sci. (USA) 82:3688-3692 (1985); Hwang et al., Proc. Natl. Acad. Sci. (USA) 77:4030-4034 (1980); EP 52,322; EP 36,676; EP 88,046; EP 143,949; EP 142,641; Japanese Pat. Appl. 83-118008; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal therapy.

For parenteral administration, the pharmaceutical composition is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation.

Generally, the formulations are prepared by contacting the components of the pharmaceutical composition uniformly and intimately with liquid carriers or finely divided solid carriers or both. Then, if necessary, the product is shaped into the desired formulation. Preferably the carrier is a parenteral carrier, more preferably a solution that is isotonic with the blood of the recipient. Examples of such carrier vehicles include water, saline, Ringer's solution, and dextrose solution. Non aqueous vehicles such as fixed oils and ethyl oleate are also useful herein, as well as liposomes. The carrier suitably contains minor amounts of additives such as substances that enhance isotonicity and chemical stability. Such materials are non-toxic to recipients at the dosages and concentrations employed, and include buffers such as phosphate, citrate, succinate, acetic acid, and other organic acids or their salts; antioxidants such as ascorbic acid; low molecular weight (less than about ten residues) (poly)peptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, manose, or dextrins; chelating agents such as EDTA; sugar alcohols such as mannitol or sorbitol; counterions such as sodium; and/or nonionic surfactants such as polysorbates, poloxamers, or PEG.

The components of the pharmaceutical composition to be used for therapeutic administration must be sterile. Sterility is readily accomplished by filtration through sterile filtration membranes (e.g., 0.2 micron membranes). Therapeutic components of the pharmaceutical composition generally are placed into a container having a sterile access port, for example, an intravenous solution bag or vial having a stopper pierceable by a hypodermic injection needle.

The components of the pharmaceutical composition ordinarily will be stored in unit or multi-dose containers, for example, sealed ampoules or vials, as an aqueous solution or as a lyophilized formulation for reconstitution. As an example of a lyophilized formulation, 10-ml vials are filled with 5 ml of sterile-filtered 1% (w/v) aqueous solution, and the resulting mixture is lyophilized. The infusion solution is prepared by reconstituting the lyophilized compound(s) using bacteriostatic Water-for-Injection.

Norrin or a functional fragment thereof to be used in accordance with the present invention may be prepared by standard (biotechnological) methods which are well known in the art. For example, a vector may be used comprising a nucleic acid molecule as defined in items (a) and (c) to (f) of the present invention herein above. The term “vector” as used herein particularly refers to plasmids, cosmids, viruses, bacteriophages and other vectors commonly used in genetic engineering. In a preferred embodiment, the vectors to be used in context of the invention are suitable for the transformation of cells, like fungal cells, cells of microorganisms such as yeast or bacterial cells, or, animal cells. In a particularly preferred embodiment such vectors are suitable for stable transformation of host cells/host tissue(s) to be used for the preparation of Norrin or a functional fragment thereof.

Accordingly, in one aspect of the invention, the vector as provided is an expression vector. Generally, expression vectors have been widely described in the literature. As a rule, they may not only contain a selection marker gene and a replication-origin ensuring replication in the host selected, but also a promoter, and in most cases a termination signal for transcription. Between the promoter and the termination signal there is in general at least one restriction site or a polylinker which enables the insertion of a nucleic acid sequence/molecule desired to be expressed. Generally, an “expression vector” is a construct that can be used to transform a selected host and provides for expression of a coding sequence, for example a nucleic acid molecule encoding Norrin or a functional fragment thereof in the selected host.

It is to be understood that when the vector to be used herein is generated by taking advantage of an expression vector known in the prior art (e.g. pACP2), a promoter suitable to be used in context of this invention (for example a βB1-crystallin promoter) may be inserted in this prior art vector. The skilled person knows how such insertion can be put into practice.

A non-limiting example of the vector to be used herein is the plasmid vector Zero Blunt comprising the nucleic acid molecule as defined in items (a) and (c) to (f) of the present invention. Further examples of vectors suitable to comprise the nucleic acid construct of the present invention to form the vector to be used in accordance with the present invention are known in the art and are, for example the pBluescript vectors (Alting-Mees, Methods Enzymol. 1992, 216:483). Typical cloning vectors to be used herein include PUC18, pBluescript SK, pGEM, pUC9, pBR322 and pGBT9. Typical expression vectors include pTRE, pCAL-n-EK, pESP-1, pOP13CAT.

In an additional embodiment, the present invention relates to a host cell comprising the nucleic acid molecule or the vector of the present invention. Preferably, the host cell of the present invention may be an animal host cell, for example, a non-human animal host cell. A non-limiting example of host cells to be used are HEK 293 EBNA cells (human embryonic kidney cells expressing Epstein-Barr nuclear antigen (EBNA)-1) which are also used in the appended example for expression of a nucleic acid molecule encoding recombinant Norrin. Accordingly, human cells are envisaged to be used as host cells in context of the present invention. As a non limiting example, the host cell of the present invention may also be an embryonic stem cell (ES cell), preferably a non-human animal ES.

Generally, the host cell to be used for the preparation of Norrin or a functional fragment thereof may be a prokaryotic or eukaryotic cell, comprising the nucleic acid molecule or the vector to be used in this context or a cell derived from such a cell and containing the nucleic acid molecule or the vector. In a preferred embodiment, the host cell comprises, i.e. is genetically modified with, the nucleic acid molecule or the vector of the invention in such a way that it contains the nucleic acid molecule as defined herein above integrated into the genome. For example, such host cell of the invention, but also the host cell of the invention in general, may be a bacterial, yeast, fungus, plant or animal cell.

In one particular aspect, the host cell of the present invention is capable to express or expresses (a) gene(s) encoding Norrin or a functional fragment thereof to be used or as defined in the present invention. An overview of examples of different corresponding expression systems to be used for generating the host cell to be used herein, is contained in Methods in Enzymology 153 (1987), 385-516, in Bitter et al. (Methods in Enzymology 153 (1987), 516-544) and in Sawers et al. (Applied Microbiology and Biotechnology 46 (1996), 1-9), Billman-Jacobe (Current Opinion in Biotechnology 7 (1996), 500-4), Hockney (Trends in Biotechnology 12 (1994), 456-463), Griffiths et al., (Methods in Molecular Biology 75 (1997), 427-440).

The transformation or genetically engineering of the host cell with a nucleic acid construct or vector according to the invention can be carried out by standard methods, as for instance described in Sambrook and Russell (2001), Molecular Cloning: A Laboratory Manual, CSH Press, Cold Spring Harbor, N.Y., USA; Methods in Yeast Genetics, A Laboratory Course Manual, Cold Spring Harbor Laboratory Press, 1990. Moreover, the host cell of the present invention is cultured in nutrient media meeting the requirements of the particular host cell used, in particular in respect of the pH value, temperature, salt concentration, aeration, antibiotics, vitamins, trace elements etc.

The conditions for culturing a host, which allow the expression of Norrin or a functional fragment thereof are known in the art to depend on the host system and the expression system/vector used in such process. The parameters to be modified in order to achieve conditions allowing the expression of a recombinant polypeptide are known in the art. Thus, suitable conditions can be determined by the person skilled in the art in the absence of further inventive input.

Once expressed, the Norrin (or a functional fragment thereof) as defined herein can be purified according to standard procedures of the art, including ammonium sulfate precipitation, affinity columns, column chromatography, gel electrophoresis and the like; see, Scopes, “Protein Purification”, Springer-Verlag, N.Y. (1982). Substantially pure polypeptides of at least about 90 to 95% homogeneity are preferred, and 98 to 99% or more homogeneity are most preferred, for pharmaceutical uses. Once purified, partially or to homogeneity as desired, the polypeptide of the invention may then be used therapeutically (including extracorporeally) or in developing and performing assay procedures. Furthermore, examples for methods for the recovery of the polypeptide of the invention from a culture are described in detail in the appended example.

Norrin or a functional fragment thereof as defined herein above may also be used in gene therapy. For example, nucleic acids comprising sequences encoding Norrin or a functional fragment thereof are administered to treat or prevent a disease associated with an increased TGF-β activity (and, in particular, an increased TGF-β level) by way of gene therapy. Gene therapy refers to therapy performed by the administration to a subject of an expressed or expressible nucleic acid. In this aspect of the invention, the nucleic acids produce their encoded protein that mediates a therapeutic effect.

Any of the methods for gene therapy available in the art can be used according to the present invention. Exemplary methods are described below.

For general reviews of the methods of gene therapy, see Goldspiel et al., Clinical Pharmacy 12:488-505 (1993); Wu and Wu, Biotherapy 3:87-95 (1991); Tolstoshev, Ann. Rev. Pharmacol. Toxicol. 32:573-596 (1993); Mulligan, Science 260:926-932 (1993); and Morgan and Anderson, Ann. Rev. Biochem. 62:191-217 (1993); May, TIBTECH 1 1(5):155-215 (1993). Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises, or alternatively consists of, nucleic acids encoding Norrin or a functional fragment thereof, said nucleic acids being part of an expression vector that expresses a gene encoding Norrin or a functional fragment thereof or chimeric proteins (e.g. fusion proteins comprising (functional) Norrin or a functional fragment thereof) in a suitable host. In particular, such nucleic acids have promoters, preferably heterologous promoters, operably linked to the antibody coding region, said promoter being inducible or constitutive, and, optionally, tissue-specific. In another particular embodiment, nucleic acid molecules are used in which the Norrin (or a functional fragment thereof) coding sequences and any other desired sequences are flanked by regions that promote homologous recombination at a desired site in the genome, thus providing for intrachromosomal expression of the Norrin encoding nucleic acids (Koller and Smithies, Proc. Natl. Acad. Sci. USA 86:8932-8935 (1989); Zijlstra et al., Nature 342:435-438 (1989).

Delivery of the nucleic acids into a patient may be either direct, in which case the patient is directly exposed to the nucleic acid or nucleic acid-carrying vectors, or indirect, in which case, cells are first transformed with the nucleic acids in vitro, then transplanted into the patient. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, J. Biol. Chem. 262:4429-4432 (1987)) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06 180; WO 92/22715; W092/203 16; W093/14188, WO 93/20221). Alternatively, the nucleic acid can be introduced intracellularly and incorporated within host cell DNA for expression, by homologous recombination (Koller and Smithies, Proc. Natl. Acad. Sci. USA 86:8932-8935 (1989); Zijlstra et al., Nature 342:435-438 (1989)).

In a specific embodiment, viral vectors that contain nucleic acid sequences encoding Norrin or a functional fragment thereof are used. For example, a retroviral vector can be used (see Miller et al., Meth. Enzymol. 217:581-599 (1993)). These retroviral vectors contain the components necessary for the correct packaging of the viral genome and integration into the host cell DNA. The nucleic acid sequences encoding Norrin (or a functional fragment thereof) to be used in gene therapy are cloned into one or more vectors, which facilitates delivery of the gene into a patient. More detail about retroviral vectors can be found in Boesen et al., Biotherapy 6:29 1-302 (1994), which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., J. Clin. Invest. 93:644-651(1994); Klein et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993).

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, Current Opinion in Genetics and Development 3:499-503 (1993) present a review of adenovirus-based gene therapy. Bout et al., Human Gene Therapy 5:3-10 (1994) demonstrated the use of adenovirus vectors to transfer genes to the respiratory epithelia of rhesus monkeys. Other instances of the use of adenoviruses in gene therapy can be found in Rosenfeld et al., Science 252:431-434 (1991); Rosenfeld et al., Cell 68:143-155 (1992); Mastrangeli et al., J. Clin. Invest. 91:225-234 (1993); PCT Publication W094/12649; and Wang, et al., Gene Therapy 2:775-783 (1995). In a preferred embodiment, adenovirus vectors are used.

Adeno-associated virus (AAV) has also been proposed for use in gene therapy (Walsh et al., Proc. Soc. Exp. Biol. Med. 204:289-300 (1993); U.S. Pat. No. 5,436,146). Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a patient.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, Meth. Enzymol. 217:599-718 (1993); Cohen et al., Meth. Enzymol. 217:718-644 (1993); Clin. Pharma. Ther. 29:69-92m (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a patient by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc. Preferably, the cell used for gene therapy is autologous to the patient.

In an embodiment in which recombinant cells are used in gene therapy, nucleic acid sequences encoding Norrin (or a functional fragment thereof) are introduced into the cells such that they are expressible by the cells or their progeny, and the recombinant cells are then administered in vivo for therapeutic effect. In a specific embodiment, stem or progenitor cells are used. Any stem and/or progenitor cells which can be isolated and maintained in vitro can potentially be used in accordance with this embodiment of the present invention (see e.g., PCT Publication WO 94/08598; Stemple and Anderson, Cell 7 1:973-985 (1992); Rheinwald, Meth. Cell Bio. 21A:229 (1980); and Pittelkow and Scott, Mayo Clinic Proc. 71:771 (1986)). In a specific embodiment, the nucleic acid to be introduced for purposes of gene therapy comprises an inducible promoter operably linked to the coding region, such that expression of the nucleic acid is controllable by controlling the presence or absence of the appropriate inducer of transcription.

The present invention is further described by reference to the following non-limiting figures and examples.

The Figures show:

FIG. 1.

FIG. 1A shows the nucleic acid sequence encoding human Norrin (SEQ ID NO: 1). FIG. 1B shows the amino acid sequence of human Norrin (SEQ ID NO: 2).

FIG. 2.

FIG. 2A shows the nucleic acid sequence encoding murine Norrin (SEQ ID NO: 3). FIG. 2B shows the amino acid sequence of murine Norrin (SEQ ID NO: 4).

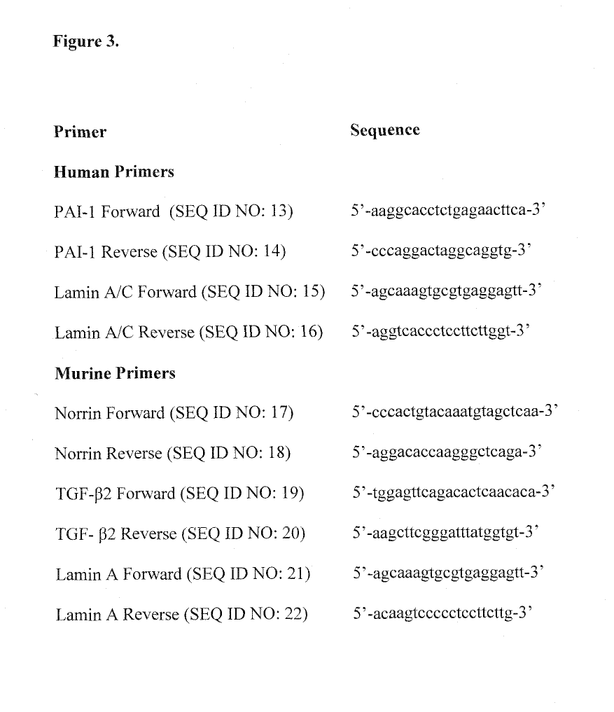

FIG. 3.

List of Primers used for quantitative PCR (SEQ ID NOs: 19 to 28).

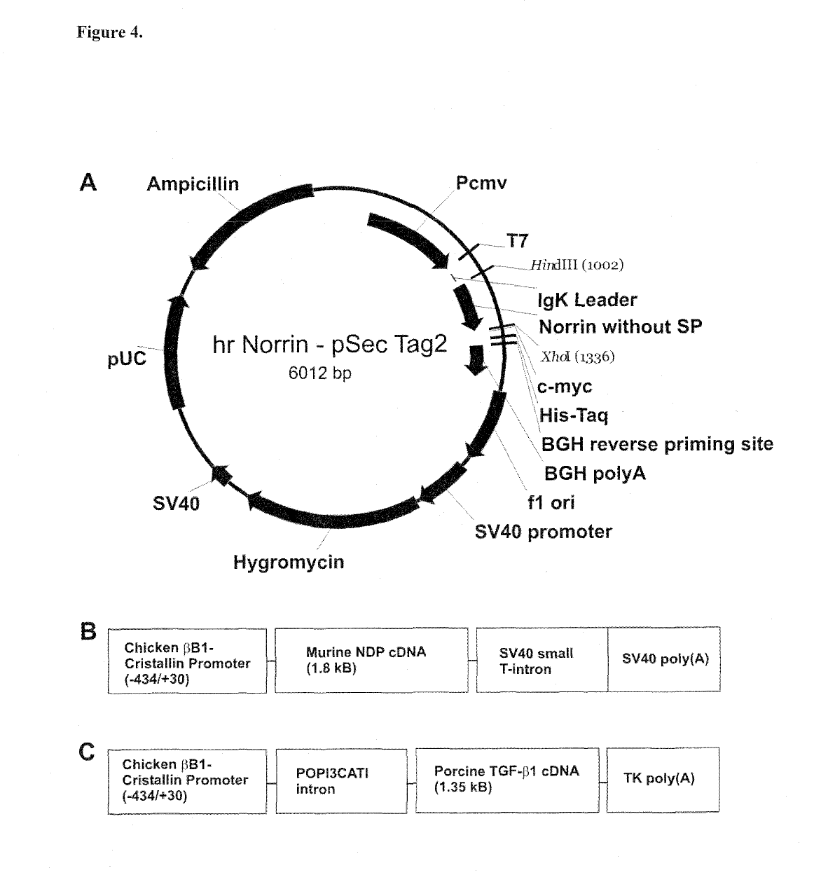

FIG. 4.

Schematic drawing of the expression vector for human recombinant norrin (hr Norrin-pSec Tag2, A) and the constructs for βB1-Norrin (B) and βB1-TGF-β1 (C) transgenic mice.

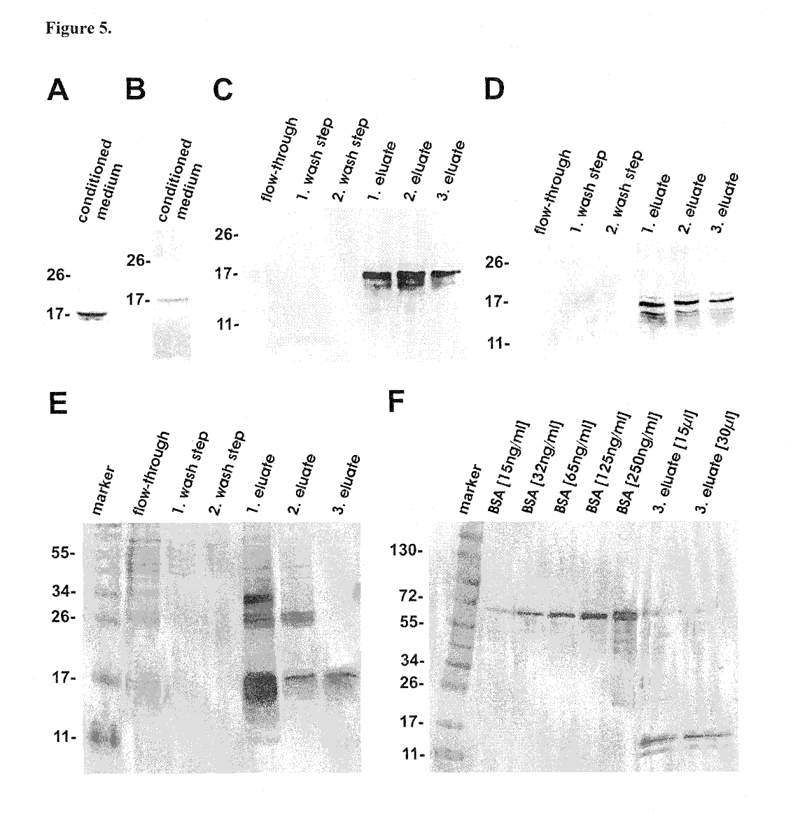

FIG. 5.

Conditioned cell culture medium of transfected HEK 293 EBNA cell line was subjected for western blot analyses (A,B). Recombinant norrin was detected with antibodies against the His (A) and the c-myc (B) epitopes.

Characterization of recombinant norrin during isolation and purification steps shows a major band at approximately 17 kDa by western blot analyses using a anti-norrin (C) and anti-His (D) antibodies and by SDS-PAGE silver staining (E,F). A slower and two faster migrating additional bands were detected by western blot analyses (C, D) and silver gel staining (F), indicating posttranslational modifications. The third eluate with a high degree of purity and protein concentration of recombinant norrin and was used in further experiments (E). Protein concentration of recombinant protein was calculated by semi-quantitative silver gel staining (F).

FIG. 6.

MELCs were incubated with TGF-β1 [1 ng/ml], recombinant norrin [40 ng/ml] and DKK-1 [100 ng/ml] for 20 hours. Cells were lysed and luciferase activity was measured by luciferase reporter assay. Shown are the means of 3 (n≦18; A) and 2 (n=28; B) independent experiments (mean±SEM). RLU, relative luciferase units.

FIG. 7.

Confluent HDMEC were incubated with norrin, TGF-β1, or the combination of both growth factors for 3 days. Total RNA was prepared and quantitative RT-PCR was performed for PAI-1 mRNA. Shown are the means of two independent experiments (mean±SEM; n=4).

FIG. 8.

HRMEC have been incubated with norrin [40 ng/ml], TGF-β1 [1 ng/ml], or the combination of both growth factors for 24 hours. Cells were fixed and the content of incorporated BrdU was measured by ELISA. Shown is the mean of two independent experiments (mean±SEM, n=12).

FIG. 9.

HRMEC were incubated with TGF-β1 [1 ng/ml], norrin [20 and 40 ng/ml], or the combination of both growth factors for three hours. The nuclear protein fraction was isolated and subjected for western blot analyses. After blotting, protein samples were stained for β-catenin. GAPDH was used as loading control. The diagram shows the mean of 3 independent experiments (mean±SEM).

FIG. 10.

Light microscopy of the retina of transgenic βB1-TGFβ1 (A, B) mice with an overexpression of TGF-β1 and double transgenic βB1-Norrin/βB1-TGFβ1 mice with an overexpression of norrin and TGF-β1 (C). At postnatal day 2 (P2), mice with an overexpression of TGF-β1 showed several pycnotic nuclei (arrows) in the retina, indicating apoptotic cell death. Apoptotic cell death leads to a progressive loss of retinal neurons that is obviously seen at postnatal day 18 (P18). Double transgenic mice that overexpress TGF-β1 and norrin (C) are protected against TGF-β1 mediated neuronal cell death and exhibit substantially more neurons than retinas of mice that only overexpress TGF-β1.

FIG. 11.

The retina of transgenic βB1-Norrin (A) and βB1-TGFβ1 (B) mice and wild type animals was prepared at postnatal day 8 (P8) and subjected to quantitative RT-PCR analyses. Shown are the mean mRNA levels of the retina from more than 4 animals (mean±SD; n=2).

The Example illustrates the invention.

### EXAMPLE

Mutual Inhibition of Transforming Growth Factor-β and Norrin

Methods

Generation and Screening of Transgenic Mice

Transgenic βB1-TGF-β1 and βB1-Norrin mice were generated as described in detail previously; see Flügel-Koch (2002) Dev Dyn 225, 111-25; Ohlmann (2005), J. Neurosci. 25, 1701-10. In brief, for generation of the βB1-TGF-β1 construct, plasmid ppK9a containing a mutated porcine TGF-β1 cDNA that ensures the secretion of bioactive TGF-β1 was kindly provided by Anita Roberts, National Cancer Institute, Bethesda, Md.; see Brunner (1989), J Biol Chem 264, 13660-13664. A BglII fragment of ppK9a containing the porcine cDNA of TGF-β1 was cloned between intron and thymidine kinase (TK) polyA sequences of the POP13/SK⁺ vector using the BamHI restriction site to obtain plasmid pER13. A −434/+30 fragment of the chicken βB1-crystallin promoter was PCR-amplified from plasmid pB434 using primers with PvuII restrictions sites at the ends and introduced into pER13 18 bp upstream of the intron sequence using a SrfI restriction site to obtain plasmid pER17-5 (FIG. 4B).

For generation of the βB1-Norrin construct, the murine cDNA of norrin was excised from plasmid pBluescript SK⁻ by EcoRI and XhoI digest as described in Berger (1996, loc. cit.) and cloned between the EcoRI and XhoI sites of plasmid pACP2 containing the simian virus 40 (SV40) polyA signal region and the SV40 small-T intron. A −434/+30 fragment of the βB1-crystallin promoter was PCR amplified from plasmid pER17-5 using primers with EcoRI and XbaI restriction sites at the ends and cloned between the EcoRI and XbaI restriction sites upstream from the murine norrin cDNA to obtain plasmid βB1-Norrin (FIG. 4C).

Both constructs were analyzed by automated sequencing. For microinjection, constructs were released from plasmid pER17-5 by digest with SpeI and XhoI, and from plasmid βB1-Norrin by digest with XbaI. Pronucleus injection and embryo transfer to obtain FVB/N transgenic βB1-TGF-β1 mice was done at the National Eye Institute Transgenic Facility (Bethesda, Md.) and for transgenic βB1-Norrin mice at the Transgenic Facility of the Albert Einstein College of Medicine (Bronx, N.Y.) as described by Wawrousek (1990), Dev Biol 137, 68.

Potential βB1-TGF-β1 transgenic mice were screened by PCR analyses using a primer pair that span from the promoter sequences to the intron of the transgene. The sequences of the primers were 5′-ACACTGATGAGCTGGCACTTCCATT-3′ (SEQ ID NO: 11) and 5′-TGTTGGCTACTTGTCTCACCATTGTA-3′ (SEQ ID NO: 12). A 506 bp DNA fragment was amplified by using the thermal cycle profile of denaturation at 94° C. for 30 sec, annealing at 55° C. for 30 sec and extension at 72° C. for 45 sec for 30 cycles. PCR analyses for βB1-Norrin transgenic mice were performed with primer pairs that span from the promoter sequences to the norrin cDNA of the transgene (5′-ACACTGATGAGGTGGCACTTCCATT-3′ (SEQ ID NO: 13) and 5′-TGCATTCCTCACAGTGACAGGAG-3′ (SEQ ID NO: 14), product length 768 bp). The thermal cycle profile was denaturation at 94° C. for 30 sec, annealing at 58° C. for 30 sec and extension at 72° C. for 1 min for 30 cycles.

Plasmid Construction for Human Recombinant Norrin

The cDNA for human norrin was obtained from RNA of human retinal pigment epithelium (RPE) cell cultures by RT-PCR using the primer pairs 5′-CCTCCCTCTGCTGTTCTTCT-3′ (SEQ ID NO: 15) and 5′-CAGTTCGCTGGCTGTGAGTA-3′ (SEQ ID NO: 16), and was cloned into plasmid Zero Blunt according to the manufacturer's instructions (Invitrogen, Karlsruhe, Germany). The sequence of human norrin cDNA was verified by automated sequencing in both directions using standard M13-forward and -reverse primers. To replace the endogenous signal peptide (SP) of human norrin, the SP was identified between amino acid 1 and 24 using the SignalP 3.0 server of the Center for Biological Sequence Analysis (Lyngby, D K http://www.cbs.dtu.dk/services/SignalP). An additional PCR with the following primer pairs 5′-GTCGAAGCTTAAAACGGACAGCTCATTCATAATG-3′ (SEQ ID NO: 17) and 5′-GGTACTCGAGAGGAATTGCATTCCTCGCA-3′ (SEQ ID NO: 18) was performed, to amplify the cDNA sequence of human norrin without the putative SP and to introduce the restriction sites of HindIII at the 5′ and of XhoI at the 3′ end of the cDNA, respectively. After digestion of the PCR product with HindIll and XhoI, the construct was ligated into the eukaryotic expression plasmid pSeqTag2 (Invitrogen) by standard techniques. At the 5′ end of the resulting plasmid, the endogenous norrin SP was replaced by the SP of the murine Ig κ-chain and at the 3′ end, sequences of the c-myc and 6× His epitopes were added before the stop codon (FIG. 4A). Finally, the sequence of the recombinant norrin cDNA was verified by automated sequencing.

Purification of Human Recombinant Norrin

Purification of recombinant norrin was performed by affinity chromatography. Heparin agarose (Sigma, Taufkirchen Germany) was washed three times with PBS and incubated in PBS for additional ten minutes. After eqilibration, heparin agarose was incubated with conditioned cell culture medium for one hour at 4° C. and loaded on empty chromatography columns (Biorad, Munich, Germany). After washing three times with PBS, bound proteins were eluted from the agarose using 1 to 2M NaCl in PBS. Eluted fractions were analyzed by Western blot analyses using antibodies against human norrin and the HisTag epitope. The purity of norrin-containing fractions was examined by silver staining of a SDS-polyacrylamide gel according to standard protocols. Fractions that were highly enriched with norrin and without detectable amounts of contaminating proteins were dialyzed overnight against PBS using a dialysing membrane with a 2-kDa cutoff (Spectra/Por, Gehrden, Germany). Protein content was measured on a semiquantitative SDS-polyacrylamid gel, and visualized by silver staining according to standard protocols.

Cell Culture

For expression of human recombinant norrin, HEK 293 EBNA cells were transfected with 2 μg of plasmid hr norrin-pSec Tag2 using lipofectamine (Invitrogen) according to manufacturer's instructions. After incubation for 4 days in DMEM containing 5% FCS, gentamycin [20 μg/ml] and genetecin (G418) [250 μg/mL], hygromycin [300 μg/mL] (all antibiotics from Invitrogen) was added for selection. Long-term cell culture was performed in spinner flasks in selection medium. For protein purification, transfected cells were cultured in medium without FCS for 3 days. Conditioned medium was collected and for recovery of the cells, FCS containing medium was added again. Human retinal microvascular endothelial cells (HRMEC, Cell Systems, Kirkland, Wash.) and human dermal microvascular endothelial cells (HDMEC, Promocell, Heidelberg) were cultured in supplemented Microvascular Endothelial Cell Growth Medium (Provitro, Berlin, Germany) containing penicillin [100 U/ml] and streptomycin [100 μg/ml]. Transfected mink lung epithelial cells (MLECs), were cultured in DMEM supplemented with 10% FCS penicillin [100 U/ml], streptomycin [100 μg/ml] and genetecin (G418) [250 μg/mL] (Invitrogen).

Protein Preparation and Western Blot Analyses

For β-catenin western blot analysis, a nuclear protein fraction was isolated. After starving overnight in cell culture medium without supplement, confluent HRMEC were incubated with Norrin [40 ng/ml], TGF-β1 [1 ng/ml], or the combination of both growth factors for three hours. Cells were harvested in PBS and pelleted by centrifugation. Supernatant was discarded and HRMEC were resuspended in hypotonic buffer (10 mM Hepes, 1.5 mM MgCl₂, 10 mM KCl, 0.2 mM PMSF, 0.5 mM DTT). After incubation on ice for ten minutes, swollen cells were dounced and nuclei were collected by centrifugation at 5000 rpm for 15 minutes. Nuclei were resuspended in low salt buffer (20 mM Hepes, 25% Glycerol, 1.5 mM MgCl₂, 0.2M KCl, 0.2M EDTA 0.2 mM PMSF, 0.5 mM DTT) and in a dropwise fashion, an equal volume of high salt buffer (20 mM Hepes, 25% Glycerol, 1.5 mM MgCl₂, 0.2M KCl, 0.2M EDTA 0.2 mM PMSF, 0.5 mM DTT) was added. After homogenization, insoluble constituents were removed by centrifugation.

Protein content was measured and up to 25 μg of nuclear proteins were subjected to SDS-PAGE. Separated proteins were transferred on a PVDF membrane (Roche, Mannheim, Germany) by semi dry blotting. After blocking with 5% low fat milk in PBS-T, the membrane was incubated overnight with a rabbit-anti-β-catenin antibody (Cell Signaling, Frankfurt am Main, Germany), diluted 1:1000 in 5% BSA in PBS-T. An HRP-conjugated chicken-anti-rabbit antibody was used as secondary antibody at a 1:2000 dilution in PBS-T with 5% BSA. Antibody labelling was visualized using the Immobilon HRP substrate (Millipore, Schwalbach, Germany) and documented with the BAS 3000 Imager work station (Fujifilm, Düsseldorf, Germany). As loading control, a HRP-conjugated anti-GAP-DH antibody was used (Rockland, Gilbertsville, Pa.).

For detection of human recombinant norrin, conditioned cell culture medium or eluted fractions of protein purification were loaded onto a 15% SDS-polyacrylamid gel. After transfer, the membrane was blocked with 2% low fat milk in PBS-T and incubated for one hour with a goat-anti-human-norrin-antibody (R&D Systems, Wiesbaden, Germany), diluted 1:1000 in PBS-T, or a rabbit-anti-His-antibody (Dianova, Hamburg, Germany), diluted 1:1000 in PBS-T or a mouse-anti-c-myc-antibody (Invitrogen), diluted 1:1000 in PBS-T. As secondary antibodies, HRP-conjugated chicken-anti-goat, chicken-anti-rabbit or chicken-anti-mouse antibodies (all Santa Cruz, Heidelberg, Germany) were used (see above).

Measurement of Bioactive TGF-β by Luciferase Activity Assay

To investigate the bioactivity of TGF-β after incubation with recombinant norrin, a well established, specific and sensitive bioassay was used; see Abe (1994), Anal Biochem 216, 276. Mink lung epithelial cells (MLECs), transfected with a luciferase reporter under the control of a TGF-β responsive truncated plasminogen activator inhibitor (PAI)-promotor fragment were seeded at a density of 2×10⁴ cells per well onto a 96-well tissue culture plate (Nunc, Wiesbaden, Germany). After attachment, MLECs were incubated with TGF-β1 [1 ng/ml] (Roche), human recombinant norrin [40 ng/ml] and/or dickkopf (DKK)-1 [100 ng/ml] (R&D Systems, Wiesbaden, Germany) for 20 hours. Cells were lysed and luciferase activity was measured as described previously with an Autolumat LB953 (Berthold, Wildbad, Germany); see Kirstein (2000), Genes to Cells 5, 661-676.

Cell Proliferation Assay

Proliferation of human retinal microvascular endothelial cells (HRMEC) was investigated by BrdU-labelling, according to the manufactures instructions (Roche). In brief, 4000 to 5000 cells were seeded per well onto 96 well culture dishes. After cell attachment, supplemented cell culture medium was replaced by BrdU-containing endothelial cell culture medium without supplement and HRMEC were incubated with Norrin [40 ng/ml], TGF-β1 [1 ng/ml] (Roche), or the combination of both growth factors. After 24 hours, cells were fixed and BrdU-labelled DNA was detected by ELISA, according to the manufacture's instructions. Calorimetric analyses were performed with an ELISA plate reader (Tecan, Crailsheim, Germany) measuring the absorption at 450 nm.

Real Time PCR Analyses

Confluent HDMEC were harvested from 35-mm cell culture dishes, and total RNA was extracted using TRIZOL (Invitrogen) according to manufacturer's recommendations. For RNA isolation, mouse retinas were homogenized in TRIZOL and the integrity of the obtained RNA was confirmed by gel electrophoresis. In addition, concentration of total RNA and purity were determined photometrically. First-strand cDNA synthesis was prepared from total RNA using the iScript cDNA Synthesis Kit (BioRad; München, Germany) according to manufacturer's instructions. Real-time PCR analyses were performed using the BioRad iQ5 Real-Time PCR Detection System. The temperature profile was denaturation at 95° C. for 10 sec and annealing and extension at 60° C. for 40 sec for 40 cycles. All PCR primers (FIG. 3; SEQ ID NO: 19 to 28) span exon-intron boundaries. For quantification, the housekeeping gene Lamin A was used simultaneously. Results were calculated using Bio-Rad iQ5 Standard-Edition (Version 2.0.148.60623) software.

Results

Protein Isolation and Characterization

Western blot analysis demonstrated that recombinant norrin with a molecular mass of approximately 17 kDa was expressed and secreted by HEK 293 EBNA cells. Staining with anti-His and anti-myc antibodies showed a major band at approximately 17 kDa in conditioned medium. In addition, a weaker faster migrating band was detected by both antibodies (FIG. 5A, B). No band was observed in cell culture medium from non-transfected control cells (data not shown).

Recombinant norrin was purified from conditioned cell culture medium using heparin agarose. Eluted protein fractions were subjected to one-dimensional (1-D) SDS-PAGE to perform either Western blot analyses or silver gel staining. SDS-PAGE silver staining showed that recombinant norrin in the third elution fraction was concentrated and had a high purity. These fractions were dialysed and used in cell culture experiments. Western blot analyses were performed using anti-Norrin and anti-His antibodies. With both antibodies, a specific major band for recombinant norrin was observed at approximately at 17 kDa. Two faster and one slower migrating bands were detected with both antibodies (FIG. 5C, D), indicating different posttranslational modifications. These additional fragments were also detected by silver gel staining (FIG. 5E, F) and in conditioned cell culture medium (FIG. 5A, B). Protein concentration of recombinant norrin was calculated by semi-quantitative SDS-PAGE silver staining using known concentrations of BSA (FIG. 5F).

Norrin Decreases TGF-β1 Mediated Luciferase Activity in MLECs

Immortalized mink lung epithelial cells (MLEC) that express the luciferase cDNA under control of a TGF-β1 sensitive PAI-1 promoter fragment (Abe, loc. cit.) were incubated with TGF-β1, recombinant norrin, or the combination of both growth factors for 20 hours. As expected, TGF-β1 caused a marked increase of luciferase activity. Recombinant norrin had no influence on the luciferase expression. Surprisingly, after co-incubation of MLECs with TGF-β1 and norrin, a marked decrease of luciferase activity was observed as compared to TGF-β1-induced activity levels (FIG. 6A).

Dickkopf (DKK)-1 is an antagonist of the frizzled (Frz) co-receptor low-density lipoprotein receptor-related protein (LRP) type 5 and 6; see Bafico (2001), Nat Cell Biol 3, 683; Zorn (2001), Curr Biol 11, R592. LRP-5 is necessary for norrin/Frz-4 mediated increase of intracellular β-catenin levels; see Xu (2004), Cell 116, 883. To investigate, whether the norrin-mediated inhibition of TGF-β is downstream of the Norrin/Frz4/LRP5-complex, MLECs were incubated with TGF-β1, norrin and DKK-1 for 20 hours. DKK-1 and human recombinant norrin had no significant influence on luciferase expression. Recombinant Norrin reduced the TGF-β1 mediated luciferase activity. This reduction was neutralized in cells that were additionally incubated with DKK-1 (FIG. 6B).

Norrin Reduces TGF-β1 Mediated PAI-1 mRNA Expression

To investigate, if norrin could influence the TGF-β1-mediated expression of PAI-1, confluent human dermal microvascular endothelial cells (HDMEC) were incubated with norrin, TGF-β1 or the combination of both growth factors, and total RNA was subjected to quantitative RT-PCR analyses. After 3 days, the mRNA of PAI-1 was slightly reduced in norrin-treated cells as compared to control cells. As expected, the incubation of TGF-β1 increased the expression of PAI-1 mRNA more than 3.5-fold. In comparison with cells that were additionally incubated with norrin, a marked decrease of PAI-1 mRNA expression of about 50% was observed (FIG. 7).

TGF-β1 Reduces the Proliferative Effect of Norrin in HRMEC

HRMEC were incubated with norrin, TGF-β1, or the combination of both growth factors for 24 hours. Cells that were treated with norrin showed a marked increase in proliferation as compared to control cells. TGF-β1 alone had no significant effect on the proliferation of HRMEC. After incubation of the cells with combined TGF-β1 and norrin, the norrin-mediated proliferation was significantly reduced (FIG. 8).

TGF-β1 Inhibits Norrin Mediated Nuclear β-Catenin Accumulation

Human retinal microvascular endothelial cells were incubated with TGF-β1, norrin, or the combination of both growth factors. In control cells, only a low signal for β-catenin was observed by western blot analyses. After three hours of incubation with norrin, a marked increase of β-catenin levels in the nuclear protein fraction of the cells was observed (FIG. 9). As expected, TGF-β1 had only a weak influence on β-Catenin translocation into the nucleus. Co-incubation of HRMEC with norrin and TGF-β1 reduced the norrin-mediated nuclear β-catenin levels markedly by nearly 50%.

Norrin Rescues the TGF-β1 Mediated Ocular Phenotype of Transgenic Mice

Because in vitro data indicated an antagonistic action of norrin and TGF-β it was elucidated whether this effect is observed in vivo as well. Therefore, transgenic mice that express norrin (βB1-Norrin) or TGF-β1 (βB1-TGFβ1) under the control of the lens specific chicken βB1-crystallin promoter were investigated. Transgenic expression of TGF-β1 in the lens induced changes in corneal development as described previously; see Flügel-Koch, loc. cit. In addition, no capillaries and an increase of neuronal apoptosis were observed in the retina (FIG. 10A, B). By contrast, mice that overexpress norrin under control of the same βB1-crystallin promoter fragment, show a marked increase of hyaloid vessels and retinal neurons; see Ohlmann (2005), J. Neurosci. 25, 1701-10. After crossbreeding of both transgenic mouse strains, the corneal phenotype observed in βB1-TGFβ1 mice was completely rescued and animals developed a normal cornea. Also a normal retina with a regular capillary network and no progressive loss of neuronal cells was observed (FIG. 10C). In summary, any structural defects caused by TGF-β1 overexpression were rescued by additional overexpression of norrin.

Transgenic Norrin and TGF-β Inhibit Their mRNA Expression in Vivo

In the eye TGF-β2 is the major expressed isoform of TGF-β. To investigate, if norrin can reduce the mRNA expression of TGF-β2 in vivo, the retina of transgenic βB1-norrin mice was prepared and subjected to quantitative RT-PCR analyses. In the retina of βB1-Norrin mice, a marked decrease of about 35% of TGF-β2 mRNA expression was detected as compared to wild-type control animals (FIG. 11A). On the other hand, the mRNA expression of norrin in the retina of βB1-TGFβ1 mice was reduced by about 95% as compared to wild-type controls (FIG. 11B).

The present invention refers to the following nucleotide and amino acid sequences:

The sequences provided herein are available in the NCBI database and can be retrieved from www.ncbi.nlm.nih.gov/sites/entrez?dh=gene; The present invention also provides techniques and methods wherein homologous sequences, and variants of the concise sequences provided herein are used. Preferably, such “variants” are genetic variants.

SEQ ID No. 1:
Nucleotide sequence encoding human Norrin.
atgagaaaac atgtactagc tgcatccttt tctatgctct ccctgctggt gataatggga gatacagaca gtaaaacgga
cagctcattc ataatggact cggaccctcg acgctgcatg aggcaccact atgtggattc tatcagtcac ccattgtaca
agtgtagctc aaagatggtg ctcctggcca ggtgcgaggg gcactgcagc caggcgtcac gctccgagcc tttggtgtcg
ttcagcactg tcctcaagca acccttccgt tcctcctgtc actgctgccg gccccagact tccaagctga aggcactgcg
gctgcgatgc tcagggggca tgcgactcac tgccacctac cggtacatcc tctcctgtca ctgcgaggaa tgcaattcct ga

The nucleotide sequence of human Norrin is disclosed in the NCBI database under accession number NM_{—}000266. The nucleotide sequence of human Norrin is also depicted in FIG. 1A.

SEQ ID No. 2:
Amino acid sequence of human Norrin.
MRKHVLAASFSMLSLLVIMGDTDSKTDSSFIMDSDPRRCMRHHYVDSISHP
LYKCSSKMVLLARCEGHCSQASRSEPLVSFSTVLKQPFRSSCHCCRPQTSK
LKALRLRCSGGMRLTATYRYILSCHCEECNS

The amino acid sequence of human Norrin is disclosed in the NCBI database under accession number NP_{—}000257. The amino acid sequence of human Norrin is also depicted in FIG. 1B.

SEQ ID No. 3:
Nucleotide sequence encoding murine Norrin.
atgagaaatc atgtactagc tgcatccatt tctatgctct ccctgctggc cataatggga gatacagaca gcaaaacaga
cagttcattt ctgatggact ctcaacgctg catgagacac cattatgtcg attctatcag tcacccactg tacaaatgta
gctcaaagat ggtgctcctg gccagatgtg aggggcactg cagccaggca tcacgctctg agcccttggt gtccttcagc
actgtcctca agcaaccttt ccgttcctcc tgtcactgct gccgacccca gacttccaag ctgaaggctc tgcgtctgcg
ctgctcaggg ggcatgcgac ttactgccac ttaccggtac atcctctcct gtcactgtga ggaatgcagc tcctga

The nucleotide sequence of murine Norrin is disclosed in the NCBI database under accession number NM_{—}010883. The nucleotide sequence of murine Norrin is also depicted in FIG. 2A.

SEQ ID No. 4:
Amino acid sequence of murine Norrin.
MRNHVLAASISMLSLLAIMGDTDSKTDSSFLMDSQRCMRHHYVDSISHPL
YKCSSKMVLLARCEGHCSQASRSEPLVSFSTVLKQPFRSSCHCCRPQTSKL
KALRLRCSGGMRLTATYRYILSCHCEECSS

The amino acid sequence of murine Norrin is disclosed in the NCBI database under accession number NP_{—}035013. The amino acid sequence of murine Norrin is also depicted in FIG. 2B.

SEQ ID No. 5:
Nucleotide sequence encoding recombinant human Norrin. Recombinant human Norrin
comprises a signal peptide of the murine Igκ chain (encoded by nucleic acid residues
1 to 63 in SEQ ID NO: 5), polylinker sequences (encoded by nucleic acid residues
64 to 102 in SEQ ID NO: 5), Norrin (encoded by human Norrin cDNA as shown in nucleic
acid residues 103 to 429 in SEQ ID NO: 5 and as shown in nucleic acid residues 73 to
402 in SEQ ID NO: 1), polylinker sequences (encoded by nucleic acid residues 430 to 444
in SEQ ID NO: 5), Flag-tag (encoded by nucleic acid residues 445 to 474), His-tag
(encoded by nucleic acid residues 490 to 507) and a stop codon.
atggagacag acacactcct gctatgggta ctgctgctct gggttccagg ttccactggt gacgcggccc agccggccag
gcgcgcgcgc cgtacgaagc ttaaaacgga cagctcattc ataatggact cggaccctcg acgctgcatg aggcaccact
atgtggattc tatcagtcac ccattgtaca agtgtagctc aaagatggtg ctcctggcca ggtgcgaggg gcactgcagc
caggcgtcac gctccgagcc tttggtgtcg ttcagcactg tcctcaagca acccttccgt tcctcctgtc actgctgccgg
ccccagactt ccaagctga aggcactgcg gctgcgatgc tcagggggca tgcgactcac tgccacctac cggtacatcc
tctcctgtca ctgcgaggaa tgcaattcct ctcgaggagg gcccgaacaa aaactcatct cagaagagg atctgaatagc
gccgtcgacc atcatcatca tcatcattga
SEQ ID No. 6:
Amino acid sequence of recombinant human Norrin. Recombinant human Norrin comprises a
signal peptide of the murine Igκ chain (amino acids 1 to 21 in SEQ ID NO: 6), polylinker
sequences (amino acids 22 to 34 in SEQ ID NO: 6), Norrin (amino acids 35 to 143 in
SEQ ID NO: 6; encoded by human Norrin cDNA and 100% homologous to human Norrin (without
endogenous signal peptide) as shown in amino acids 25 to 133 in SEQ ID NO: 2),
polylinker sequences (amino acids 144 to 148 in SEQ ID NO: 6), Flag-tag (amino acids
149 to 158 in SEQ ID NO: 6), His-tag (amino acids 164 to 169 in SEQ ID NO: 6).
METDTLLLWVLLLWVPGSTGDAAQPARRARRTKLKTDSSFIMDSDPRRCMRHHYVDSISHPLYKCSSKMVLLARCEGHCSQASRSEP
LVSFSTVLKQPFRSSCHCCRPQTSKLKALRLRCSGGMRLTATYRYILSCHCEECNSSRGGPEQKLISEEDLNSAVDHHHHHH
SEQ ID No. 7:
Nucleotide sequence encoding human TGF-β1.
atgccgccct ccgggctgcg gctgctgccg ctgctgctac cgctgctgtg gctactggtg ctgacgcctg gccggccggc
cgcgggacta tccacctgca agactatcga catggagctg gtgaagcgga agcgcatcga ggccatccgc ggccagatcc
tgtccaagct gcggctcgcc agccccccga gccaggggga ggtgccgccc ggcccgctgc ccgaggccgt gctcgccctg
tacaacagca cccgcgaccg ggtggccggg gagagtgcag aaccggagcc cgagcctgag gccgactact acgccaagga
ggtcacccgc gtgctaatgg tggaaaccca caacgaaatc tatgacaagt tcaagcagag tacacacagc atatatatgt
tcttcaacac atcagagctc cgagaagcgg tacctgaacc cgtgttgctc tcccgggcag agctgcgtct gctgaggctc
aagttaaaag tggagcagca cgtggagctg taccagaaat acagcaacaa ttcctggcga tacctcagca accggctgct
ggcacccagc gactcgccag agtggttatc ttttgatgtc accggagttg tgcggcagtg gttgagccgt ggaggggaaa
ttgagggctt tcgccttagc gcccactgct cctgtgacag cagggataac acactgcaag tggacatcaa cgggttcact
accggccgcc gaggtgacct ggccaccatt catggcatga accggccttt cctgcttctc atggccaccc cgctggagag
ggcccagcat ctgcaaagct cccggcaccg ccgagccctg gacaccaact attgcttcag ctccacggag aagaactgct
gcgtgcggca gctgtacatt gacttccgca aggacctcgg ctggaagtgg atccacgagc ccaagggcta ccatgccaac
ttctgcctcg ggccctgccc ctacatttgg agcctggaca cgcagtacag caaggtcctg gccctgtaca accagcataa
cccgggcgcc tcggcggcgc cgtgctgcgt gccgcaggcg ctggagccgc tgcccatcgt gtactacgtg ggccgcaagc
ccaaggtgga gcagctgtcc aacatgatcg tgcgctcctg caagtgcagc tga

The nucleotide sequence of human TGF-β1 is disclosed in the NCBI database under accession number NM_{—}000660.

SEQ ID No. 8:
Amino acid sequence of human TGF-β1.
MPPSGLRLLPLLLPLLWLLVLTPGRPAAGLSTCKTIDMELVKRKRIEAI
RGQILSKLRLASPPSQGEVPPGPLPEAVLALYNSTRDRVAGESAEPEPE
PEADYYAKEVTRVLMVETHNEIYDKFKQSTHSIYMFFNTSELREAVPEP
VLLSRAELRLLRLKLKVEQHVELYQKYSNNSWRYLSNRLLAPSDSPEWL
SFDVTGVVRQWLSRGGEIEGFRLSAHCSCDSRDNTLQVDINGFTTGRRG
DLATIHGMNRPFLLLMATPLERAQHLQSSRHRRALDTNYCFSSTEKNCC
VRQLYIDFRKDLGWKWIHEPKGYHANFCLGPCPYIWSLDTQYSKVLALY
NQHNPGASAAPCCVPQALEPLPIVYYVGRKPKVEQLSNMIVRSCKCS

The amino acid sequence of human TGF-β1 is disclosed in the NCBI database under accession number NP_{—}000651

SEQ ID No. 9:
Nucleotide sequence encoding human TGF-β2.
atgcactact gtgtgctgag cgcttttctg atcctgcatc tggtcacggt cgcgctcagc ctgtctacct gcagcacact
cgatatggac cagttcatgc gcaagaggat cgaggcgatc cgcgggcaga tcctgagcaa gctgaagctc accagtcccc
cagaagacta tcctgagccc gaggaagtcc ccccggaggt gatttccatc tacaacagca ccagggactt gctccaggag
aaggcgagcc ggagggcggc cgcctgcgag cgcgagagga gcgacgaaga gtactacgcc aaggaggttt acaaaataga
catgccgccc ttcttcccct ccgaaaatgc catcccgccc actttctaca gaccctactt cagaattgtt cgatttgacg
tctcagcaat ggagaagaat gcttccaatt tggtgaaagc agagttcaga gtctttcgtt tgcagaaccc aaaagccaga
gtgcctgaac aacggattga gctatatcag attctcaagt ccaaagattt aacatctcca acccagcgct acatcgacag
caaagttgtg aaaacaagag cagaaggcga atggctctcc ttcgatgtaa ctgatgctgt tcatgaatgg cttcaccata
aagacaggaa cctgggattt aaaataagct tacactgtcc ctgctgcact tttgtaccat ctaataatta catcatccca
aataaaagtg aagaactaga agcaagattt gcaggtattg atggcacctc cacatatacc agtggtgatc agaaaactat
aaagtccact aggaaaaaaa acagtgggaa gaccccacat ctcctgctaa tgttattgcc ctcctacaga cttgagtcac
aacagaccaa ccggcggaag aagcgtgctt tggatgcggc ctattgcttt agaaatgtgc aggataattg ctgcctacgt
ccactttaca ttgatttcaa gagggatcta gggtggaaat ggatacacga acccaaaggg tacaatgcca acttctgtgc
tggagcatgc ccgtatttat ggagttcaga cactcagcac agcagggtcc tgagcttata taataccata aatccagaag
catctgcttc tccttgctgc gtgtcccaag atttagaacc tctaaccatt ctctactaca ttggcaaaac acccaagatt
gaacagcttt ctaatatgat tgtaaagtct tgcaaatgca gctaa

The nucleotide sequence of human TGF-β2 is disclosed in the NCBI database under accession number NM_{—}003238.

SEQ ID No. 10:
Amino acid sequence of human TGF-β2.
MHYCVLSAFLILHLVTVALSLSTCSTLDMDQFMRKRIEAIRGQILSKLK
LTSPPEDYPEPEEVPPEVISIYNSTRDLLQEKASRRAAACERERSDEEY
YAKEVYKIDMPPFFPSENAIPPTFYRPYFRIVRFDVSAMEKNASNLVKA
EFRVFRLQNPKARVPEQRIELYQILKSKDLTSPTQRYIDSKVVKTRAEG
EWLSFDVTDAVHEWLHHKDRNLGFKISLHCPCCTFVPSNNYIIPNKSEE
LEARFAGIDGTSTYTSGDQKTIKSTRKKNSGKTPHLLLMLLPSYRLESQ
QTNRRKKRALDAAYCFRNVQDNCCLRPLYIDFKRDLGWKWIHEPKGYNA
NFCAGACPYLWSSDTQHSRVLSLYNTINPEASASPCCVSQDLEPLTILY
YIGKTPKIEQLSNMIVKSCKCS

The amino acid sequence of human TGF-β2 is disclosed in the NCBI database under accession number NP_{—}003229

SEQ ID No. 11:
Nucleotide sequence of primer for screening
potential βB1-TGF-β1 transgenic mice.
ACACTGATGAGCTGGCACTTCCATT
SEQ ID No. 12:
Nucleotide sequence of primer for screening
potential βB1-TGF-β1 transgenic mice.
TGTTGGCTACTTGTCTCACCATTGTA
SEQ ID No. 13:
Nucleotide sequence of primer for screening
potential βB1-Nonin transgenic mice.
ACACTGATGAGGTGGCACTTCCATT
SEQ ID No. 14:
Nucleotide sequence of primer for screening
potential βB1-Norrin transgenic mice.
TGCATTCCTCACAGTGACAGGAG
SEQ ID No. 15:
Nucleotide sequence of primer for amplification of
cDNA of human Norrin.
CCTCCCTCTGCTGTTCTTCT
SEQ ID No. 16:
Nucleotide sequence of primer for amplification of
cDNA of human Norrin.
CAGTTCGCTGGCTGTGAGTA
SEQ ID No. 17:
Nucleotide sequence of primer for amplification of
cDNA of human Norrin.
GTCGAAGCTTAAAACGGACAGCTCATTCATAATG
SEQ ID No. 18:
Nucleotide sequence of primer for amplification of
cDNA of human Norrin.
GGTACTCGAGAGGAATTGCATTCCTCGCA
SEQ ID No. 19:
Nucleotide sequence of forward primer for
quantitative PCR of human PAI-1.
AAGGCACCTCTGAGAACTTCA
SEQ ID No. 20:
Nucleotide sequence of reverse primer for
quantitative PCR of human PAI-1.
CCCAGGACTAGGCAGGTG
SEQ ID No. 21:
Nucleotide sequence of forward primer for
quantitative PCR of human Lamin A/C.
AGCAAAGTGCGTGAGGAGTT
SEQ ID No. 22:
Nucleotide sequence of reverse primer for
quantitative PCR of human Lamin A/C.
AGGTCACCCTCCTTCTTGGT
SEQ ID No. 23:
Nucleotide sequence of forward primer for
quantitative PCR of murine Norrin.
CCCACTGTACAAATGTAGCTCAA
SEQ ID No. 24:
Nucleotide sequence of reverse primer for
quantitative PCR of murine Norrin.
AGGACACCAAGGGCTCAGA
SEQ ID No. 25:
Nucleotide sequence of forward primer for
quantitative PCR of murine TGF-β2.
TGGAGTTCAGACACTCAACACA
SEQ ID No. 26:
Nucleotide sequence of reverse primer for
quantitative PCR of murine TGF-β2.
AAGCTTCGGGATTTATGGTGT
SEQ ID No. 27:
Nucleotide sequence of forward primer for
quantitative PCR of murine Lamin A.
AGCAAAGTGCGTGAGGAGTT
SEQ ID No. 28:
Nucleotide sequence of reverse primer for
quantitative PCR of murine Lamin A.
ACAAGTCCCCCTCCTTCTTG

1. (canceled)

2. A Norrin, or a functional fragment thereof, wherein Norrin is selected from the group consisting of
(a) a polypeptide comprising an amino acid encoded by a nucleic acid molecule having the nucleic acid sequence as depicted in SEQ ID NO: 1, the nucleic acid sequence comprising nucleic acid residues 4 to 402 in SEQ ID NO: 1 or the nucleic acid sequence comprising nucleic acid residues 73 to 402 in SEQ ID NO: 1;
(b) a polypeptide having an amino acid sequence as depicted in SEQ ID NO:2, an amino acid sequence comprising amino acids 2 to 133 in SEQ ID NO:2 or an amino acid sequence comprising amino acids 25 to 133 in SEQ ID NO:2;
(c) a polypeptide encoded by a nucleic acid molecule encoding a peptide having an amino acid sequence as depicted in SEQ ID NO:2, an amino acid sequence comprising amino acids 2 to 133 in SEQ ID NO:2 or an amino acid sequence comprising amino acids 25 to 133 in SEQ ID NO:2;
(d) a polypeptide comprising an amino acid encoded by a nucleic acid molecule hybridizing under stringent conditions to the complementary strand of nucleic acid molecules as defined in (a) or (c) and encoding a functional Norrin or a functional fragment thereof;
(e) a polypeptide having at least 60% homology to the polypeptide of any one of (a) to (d), whereby said polypeptide is a functional Norrin or a functional fragment thereof; and
(f) a polypeptide comprising an amino acid encoded by a nucleic acid molecule being degenerate as a result of the genetic code to the nucleotide sequence of a nucleic acid molecule as defined in (a), (c) and (d).

3. The Norrin of claim 2, wherein said polypeptide further comprises a signal peptide of the murine Igκ chain.

4. The Norrin of claim 3, wherein said signal peptide comprises amino acids 1 to 21 in SEQ ID NO:6.

5. The Norrin of claim 4, wherein said Norrin is a polypeptide having an amino acid sequence as depicted in SEQ ID NO:6.

6. A Method for treating or preventing a disease associated with an increased TGF-beta activity comprising the administration of an effective amount of Norrin or a functional fragment thereof as defined in claim 2 to a subject in need of such a treatment or prevention.

7. The method of claim 6, wherein said subject is a human.

8. The Norrin of claim 2 wherein said disease associated with an increased TGF-beta activity is a fibrotic disease or a proliferative disease.

9. The Norrin of claim 8 or the method of claim 8, wherein said fibrotic disease is selected from the group consisting of chronic pancreatitis, pancreatic fibrosis, fibrosis of the conjunctiva, cystic fibrosis, injection fibrosis, endomyocardial fibrosis, mediastinal fibrosis, myleofibrosis, retroperitoneal fibrosis, nephrogenic systemic fibrosis, diabetic nephropathy, post-vasectomy pain syndrome, rheumatoid arthritis, fibrosis of the lung, liver fibrosis, cirrhosis, dermal keloids, scleroderma, excessive scarring, fibrosis of the kidneys, glomerulosclerosis of the kidneys, remodeling during myocardial infarct healing with shortening and thickening of the infracted segment, failure after filtrating glaucoma surgery, glaucoma and cardiomyopathy with increased TGF-beta level.

10. The Norrin of claim 9, wherein said fibrosis of the lung is selected from the group consisting of Acute Respiratory Distress Syndrome (ARDS), chronic obstructive pulmonary disease (COPD), idiopathic pulmonary fibrosis, asbestosis, progressive massive fibrosis, drug-induced lung fibrosis, fibrosis resulting from pulmonary hypertension and asthma.

11. The Norrin of claim 8, wherein said proliferative disease is a cancerous disease.

12. The Norrin of claim 11, wherein said cancerous disease is selected from the group of malignant melanoma, malignant glioma, malignant tumors of the central nervous system (CNS), pancreas carcinoma, colorectal carcinoma, non-small cell lung cancer, prostate carcinoma, hematological malignancies, hepatocellular carcinoma, renal cell carcinoma, cutaneous squamous cell carcinomas, esophageal carcinoma and breast carcinoma.

13. The Norrin of claim 2, wherein said disease is associated with an increased TGF-beta 1 and/or TGF-beta 2 activity.

14. (canceled)

15. The method of claim 6, wherein said disease associated with an increased TGF-beta activity is a fibrotic disease or a proliferative disease.

16. The method of claim 15, wherein said fibrotic disease is selected from the group consisting of chronic pancreatitis, pancreatic fibrosis, fibrosis of the conjunctiva, cystic fibrosis, injection fibrosis, endomyocardial fibrosis, mediastinal fibrosis, myleofibrosis, retroperitoneal fibrosis, nephrogenic systemic fibrosis, diabetic nephropathy, post-vasectomy pain syndrome, rheumatoid arthritis, fibrosis of the lung, liver fibrosis, cirrhosis, dermal keloids, scleroderma, excessive scarring, fibrosis of the kidneys, glomerulosclerosis of the kidneys, remodeling during myocardial infarct healing with shortening and thickening of the infracted segment, failure after filtrating glaucoma surgery, glaucoma and cardiomyopathy with increased TGF-beta level.

17. The method of claim 16, wherein said fibrosis of the lung is selected from the group consisting of Acute Respiratory Distress Syndrome (ARDS), chronic obstructive pulmonary disease (COPD), idiopathic pulmonary fibrosis, asbestosis, progressive massive fibrosis, drug-induced lung fibrosis, fibrosis resulting from pulmonary hypertension and asthma.

18. The method of claim 15, wherein said proliferative disease is a cancerous disease.

19. The method of claim 18, wherein said cancerous disease is selected from the group of malignant melanoma, malignant glioma, malignant tumors of the central nervous system (CNS), pancreas carcinoma, colorectal carcinoma, non-small cell lung cancer, prostate carcinoma, hematological malignancies, hepatocellular carcinoma, renal cell carcinoma, cutaneous squamous cell carcinomas, esophageal carcinoma and breast carcinoma.

20. The method of claim 6, wherein said disease is associated with an increased TGF-beta 1 and/or TGF-beta 2 activity.


SEQUENCE LISTING (28 sequences)

SEQ ID NO: 1 - DNA, 402 bp, homo sapiens
atgagaaaacatgtactagctgcatccttttctatgctctccctgctggtgataatgggagatacagacagtaaaacggacagctcattcataatggactcggaccctcgacgctgcatgaggcaccactatgtggattctatcagtcacccattgtacaagtgtagctcaaagatggtgctcctggccaggtgcgaggggcactgcagccaggcgtcacgctccgagcctttggtgtcgttcagcactgtcctcaagcaacccttccgttcctcctgtcactgctgccggccccagacttccaagctgaaggcactgcggctgcgatgctcagggggcatgcgactcactgccacctaccggtacatcctctcctgtcactgcgaggaatgcaattcctga

SEQ ID NO: 2 - protein, 133 aa, homo sapiens
MRKHVLAASFSMLSLLVIMGDTDSKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNS

SEQ ID NO: 3 - DNA, 396 bp, mus musculus
atgagaaatcatgtactagctgcatccatttctatgctctccctgctggccataatgggagatacagacagcaaaacagacagttcatttctgatggactctcaacgctgcatgagacaccattatgtcgattctatcagtcacccactgtacaaatgtagctcaaagatggtgctcctggccagatgtgaggggcactgcagccaggcatcacgctctgagcccttggtgtccttcagcactgtcctcaagcaacctttccgttcctcctgtcactgctgccgaccccagacttccaagctgaaggctctgcgtctgcgctgctcagggggcatgcgacttactgccacttaccggtacatcctctcctgtcactgtgaggaatgcagctcctga

SEQ ID NO: 4 - protein, 131 aa, mus musculus
MRNHVLAASISMLSLLAIMGDTDSKTDSSFLMDSQRCMRHHYVDSISHPLYKCSSKMVLLARCEGHCSQASRSEPLVSFSTVLKQPFRSSCHCCRPQTSKLKALRLRCSGGMRLTATYRYILSCHCEECSS

SEQ ID NO: 5 - DNA, 510 bp, artificial
  /note=“Description of artificial sequence recombinant human Norrin”
atggagacagacacactcctgctatgggtactgctgctctgggttccaggttccactggtgacgcggcccagccggccaggcgcgcgcgccgtacgaagcttaaaacggacagctcattcataatggactcggaccctcgacgctgcatgaggcaccactatgtggattctatcagtcacccattgtacaagtgtagctcaaagatggtgctcctggccaggtgcgaggggcactgcagccaggcgtcacgctccgagcctttggtgtcgttcagcactgtcctcaagcaacccttccgttcctcctgtcactgctgccggccccagacttccaagctgaaggcactgcggctgcgatgctcagggggcatgcgactcactgccacctaccggtacatcctctcctgtcactgcgaggaatgcaattcctctcgaggagggcccgaacaaaaactcatctcagaagaggatctgaatagcgccgtcgaccatcatcatcatcatcattga

SEQ ID NO: 6 - protein, 169 aa, artificial
  /note=“Description of artificial sequence recombinant human Norrin”
METDTLLLWVLLLWVPGSTGDAAQPARRARRTKLKTDSSFIMDSDPRRCMRHHYVDSISHPLYKCSSKMVLLARCEGHCSQASRSEPLVSFSTVLKQPFRSSCHCCRPQTSKLKALRLRCSGGMRLTATYRYILSCHCEECNSSRGGPEQKLISEEDLNSAVDHHHHHH

SEQ ID NO: 7 - DNA, 1173 bp, homo sapiens
atgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctga

SEQ ID NO: 8 - protein, 390 aa, homo sapiens
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSSRHRRALDTNYCFSSTEKNCCVRQLYIDFRKDLGWKWIHEPKGYHANFCLGPCPYIWSLDTQYSKVLALYNQHNPGASAAPCCVPQALEPLPIVYYVGRKPKVEQLSNMIVRSCKCS

SEQ ID NO: 9 - DNA, 1245 bp, homo sapiens
atgcactactgtgtgctgagcgcttttctgatcctgcatctggtcacggtcgcgctcagcctgtctacctgcagcacactcgatatggaccagttcatgcgcaagaggatcgaggcgatccgcgggcagatcctgagcaagctgaagctcaccagtcccccagaagactatcctgagcccgaggaagtccccccggaggtgatttccatctacaacagcaccagggacttgctccaggagaaggcgagccggagggcggccgcctgcgagcgcgagaggagcgacgaagagtactacgccaaggaggtttacaaaatagacatgccgcccttcttcccctccgaaaatgccatcccgcccactttctacagaccctacttcagaattgttcgatttgacgtctcagcaatggagaagaatgcttccaatttggtgaaagcagagttcagagtctttcgtttgcagaacccaaaagccagagtgcctgaacaacggattgagctatatcagattctcaagtccaaagatttaacatctccaacccagcgctacatcgacagcaaagttgtgaaaacaagagcagaaggcgaatggctctccttcgatgtaactgatgctgttcatgaatggcttcaccataaagacaggaacctgggatttaaaataagcttacactgtccctgctgcacttttgtaccatctaataattacatcatcccaaataaaagtgaagaactagaagcaagatttgcaggtattgatggcacctccacatataccagtggtgatcagaaaactataaagtccactaggaaaaaaaacagtgggaagaccccacatctcctgctaatgttattgccctcctacagacttgagtcacaacagaccaaccggcggaagaagcgtgctttggatgcggcctattgctttagaaatgtgcaggataattgctgcctacgtccactttacattgatttcaagagggatctagggtggaaatggatacacgaacccaaagggtacaatgccaacttctgtgctggagcatgcccgtatttatggagttcagacactcagcacagcagggtcctgagcttatataataccataaatccagaagcatctgcttctccttgctgcgtgtcccaagatttagaacctctaaccattctctactacattggcaaaacacccaagattgaacagctttctaatatgattgtaaagtcttgcaaatgcagctaa

SEQ ID NO: 10 - protein, 414 aa, homo sapiens
MHYCVLSAFLILHLVTVALSLSTCSTLDMDQFMRKRIEAIRGQILSKLKLTSPPEDYPEPEEVPPEVISIYNSTRDLLQEKASRRAAACERERSDEEYYAKEVYKIDMPPFFPSENAIPPTFYRPYFRIVRFDVSAMEKNASNLVKAEFRVFRLQNPKARVPEQRIELYQILKSKDLTSPTQRYIDSKVVKTRAEGEWLSFDVTDAVHEWLHHKDRNLGFKISLHCPCCTFVPSNNYIIPNKSEELEARFAGIDGTSTYTSGDQKTIKSTRKKNSGKTPHLLLMLLPSYRLESQQTNRRKKRALDAAYCFRNVQDNCCLRPLYIDFKRDLGWKWIHEPKGYNANFCAGACPYLWSSDTQHSRVLSLYNTINPEASASPCCVSQDLEPLTILYYIGKTPKIEQLSNMIVKSCKCS

SEQ ID NO: 11 - DNA, 25 bp, artificial
  /note=“Description of artificial sequence primer for screening”
acactgatgagctggcacttccatt

SEQ ID NO: 12 - DNA, 26 bp, artificial
  /note=“Description of artificial sequence primer for screening”
tgttggctacttgtctcaccattgta

SEQ ID NO: 13 - DNA, 25 bp, artificial
  /note=“Description of artificial sequence primer for screening”
acactgatgaggtggcacttccatt

SEQ ID NO: 14 - DNA, 23 bp, artificial
  /note=“Description of artificial sequence primer for screening”
tgcattcctcacagtgacaggag

SEQ ID NO: 15 - DNA, 20 bp, artificial
  /note=“Description of artificial sequence primer for screening”
cctccctctgctgttcttct

SEQ ID NO: 16 - DNA, 20 bp, artificial
  /note=“Description of artificial sequence primer for screening”
cagttcgctggctgtgagta

SEQ ID NO: 17 - DNA, 34 bp, artificial
  /note=“Description of artificial sequence primer for screening”
gtcgaagcttaaaacggacagctcattcataatg

SEQ ID NO: 18 - DNA, 29 bp, artificial
  /note=“Description of artificial sequence primer for screening”
ggtactcgagaggaattgcattcctcgca

SEQ ID NO: 19 - DNA, 21 bp, artificial
  /note=“Description of artificial sequence primer for screening”
aaggcacctctgagaacttca

SEQ ID NO: 20 - DNA, 18 bp, artificial
  /note=“Description of artificial sequence primer for screening”
cccaggactaggcaggtg

SEQ ID NO: 21 - DNA, 20 bp, artificial
  /note=“Description of artificial sequence primer for screening”
agcaaagtgcgtgaggagtt

SEQ ID NO: 22 - DNA, 20 bp, artificial
  /note=“Description of artificial sequence primer for screening”
aggtcaccctccttcttggt

SEQ ID NO: 23 - DNA, 23 bp, artificial
  /note=“Description of artificial sequence primer for screening”
cccactgtacaaatgtagctcaa

SEQ ID NO: 24 - DNA, 19 bp, artificial
  /note=“Description of artificial sequence primer for screening”
aggacaccaagggctcaga

SEQ ID NO: 25 - DNA, 22 bp, artificial
  /note=“Description of artificial sequence primer for screening”
tggagttcagacactcaacaca

SEQ ID NO: 26 - DNA, 21 bp, artificial
  /note=“Description of artificial sequence primer for screening”
aagcttcgggatttatggtgt

SEQ ID NO: 27 - DNA, 20 bp, artificial
  /note=“Description of artificial sequence primer for screening”
agcaaagtgcgtgaggagtt

SEQ ID NO: 28 - DNA, 20 bp, artificial
  /note=“Description of artificial sequence primer for screening”
acaagtccccctccttcttg